(19) United States
(12) Patent Application Publication
(10) Pub. No.: US 2012/0252855 A1
(43) Pub. Date: Oct. 4, 2012

(54) Methods For Treating Degenerative Diseases/Injuries

(76) Inventors: Connie ERICKSON-MILLER, Collegeville, PA (US); Julian JENKINS, Collegeville, PA (US)

(21) Appl. No.: 13/409,324

(22) Filed: Mar. 1, 2012

Related U.S. Application Data
(63) Continuation of application No. 12/256,669, filed on Oct. 23, 2008, now abandoned.
Application No. 12/256,669 is a continuation-in-part of application No. 10/554,811, filed on Nov. 10, 2006.
(60) Provisional application No. 60/556,390, filed on Mar. 25, 2004.
(60) Provisional application No. 60/554,581, filed on Mar. 19, 2004.
(60) Provisional application No. 60/549,977, filed on Mar. 4, 2004.
(60) Provisional application No. 60/495,034, filed on Aug. 14, 2003.
(60) Provisional application No. 60/471,554, filed on May 19, 2003.
(60) Provisional application No. 60/466,540, filed on Apr. 29, 2003.

(51) Int. Cl. A61K 31/4152; A61P 9/00; A61P 9/10; A61K 31/4155

(52) U.S. Cl. 514/381; 514/404

(57) ABSTRACT

Invented is a method of treating cardiovascular disease/injury, in a mammal, including a human, in need thereof which comprises the administration of a therapeutically effective amount of a non-peptide TPO receptor agonist to such mammal.

This application is a continuation of U.S. application Ser. No. 12/256,669, fled Oct. 23, 2008, which is a Continuation-in-Part of U.S. application Ser. No. 10/554,811, filed on Nov. 10, 2006, which is a 371 of International Application No. PCT/US2004/013468 filed Apr. 29, 2004, which claims the benefit of U.S. Provisional Application Nos. 60/556,390 filed Mar. 25, 2004; 60/554,581 filed Mar. 19, 2004; 60/549,977 filed Mar. 4, 2004; 60/495,034 filed Aug. 14, 2003; 60/471,554 filed May 19, 2003 and 60/466,540 filed Apr. 29, 2003.

### FIELD OF THE INVENTION

This invention relates to non-peptide thrombopoietin (TPO) receptor agonists and their use in the treatment of degenerative diseases/injuries.

### BACKGROUND OF THE INVENTION

Thrombopoietin (TPO) has been shown to be the main humoral regulator in situations involving thrombocytopenia. See, e.g., Metcalf Nature 369:519-520 (1994). TPO has been shown in several studies to increase platelet counts, increase platelet size, and increase isotope incorporation into platelets of recipient animals. Because platelets (thrombocytes) are necessary for blood clotting and when their numbers are very low a patient is at risk of death from catastrophic hemorrhage, TPO is considered to have potential useful applications in both the diagnosis and the treatment of various hematological disorders, for example, diseases primarily due to platelet defects. In addition, studies have provided a basis for the projection of efficacy of TPO therapy in the treatment of thrombocytopenia, and particularly thrombocytopenia resulting from chemotherapy, radiation therapy, or bone marrow transplantation as treatment for cancer or lymphoma. See e.g., McDonald (1992) Am. J. Ped. Hematology/Oncology 14: 8-21 (1992).

The slow recovery of platelet levels in patients suffering from thrombocytopenia is a serious problem, and has lead to the search for small molecule non-peptide TPO receptor agonists that are able to accelerate platelet regeneration. (e.g. see, International Application Number PCT/US01/16863, having International Filing Date May 24, 2001).

However, non-peptide TPO receptor agonists are not known to have a beneficial effect in the treatment of degenerative diseases/injuries.

It would be desirable to provide compounds which allow for the treatment of degenerative diseases/injuries.

The present invention relates to novel therapeutic uses of a known class of compounds, non-peptide TPO receptor agonists. The present invention concerns a method for treating degenerative diseases/injuries in a mammal in need of such treatment.

As disclosed herein it has unexpectedly been discovered that non-peptide TPO receptor agonist compounds are useful in treating degenerative diseases/injuries.

As disclosed herein it has unexpectedly been discovered that the in vivo administration of a non-peptide TPO receptor agonist is useful in treating degenerative diseases/injuries.

As disclosed herein it has unexpectedly been discovered that non-peptide TPO receptor agonists increase the survival of stem cells to a therapeutic extent.

As disclosed herein it has unexpectedly been discovered that non-peptide TPO receptor agonists stimulate the production of stem cells to a therapeutic extent.

As disclosed herein it has unexpectedly been discovered that non-peptide TPO receptor agonists increase the number of stem cells to a therapeutic extent.

As disclosed herein it has unexpectedly been discovered that non-peptide TPO receptor agonists increase stem cell longevity to a therapeutic extent.

As disclosed herein it has unexpectedly been discovered that the in vivo administration of a non-peptide TPO receptor agonist increases the survival of stem cells to a therapeutic extent.

As disclosed herein it has unexpectedly been discovered that the in vivo administration of a non-peptide TPO receptor agonist stimulates the production of stem cells to a therapeutic extent.

As disclosed herein it has unexpectedly been discovered that the in vivo administration of a non-peptide TPO receptor agonist increases stem cell function to a therapeutic extent.

As disclosed herein it has unexpectedly been discovered that the in vivo administration of a non-peptide TPO receptor agonist increases stem cell longevity to a therapeutic extent.

### SUMMARY OF THE INVENTION

This invention relates to a method of treating a degenerative disease/injury in a mammal, including a human, in need thereof which comprises administering to such mammal a therapeutically effective amount of a non-peptide TPO receptor agonists.

This invention also relates to the discovery that non-peptide TPO receptor agonists are effective in the treatment of degenerative diseases/injuries.

This invention also relates to the discovery that non-peptide TPO receptor agonists increase the survival of stem cells to a therapeutic extent.

This invention also relates to the discovery that non-peptide TPO receptor agonists stimulate the production of stem cells to a therapeutic extent.

This invention also relates to the discovery that non-peptide TPO receptor agonists increase the number of stem cells to a therapeutic extent.

This invention also relates to the discovery that non-peptide TPO receptor agonists increase stem cell longevity to a therapeutic extent.

This invention also relates to the discovery that the in vivo administration of a non-peptide TPO receptor agonist increases the survival of stem cells to a therapeutic extent.

This invention also relates to the discovery that the in vivo administration of a non-peptide TPO receptor agonist stimulates the production of stem cells to a therapeutic extent.

This invention also relates to the discovery that the in vivo administration of a non-peptide TPO receptor agonist increases stem cell function to a therapeutic extent.

This invention also relates to the discovery that the in vivo administration of a non-peptide TPO receptor agonist increases stem cell longevity to a therapeutic extent.

Included among the non-peptide TPO receptor agonists of the invention are compounds of Formula (I):

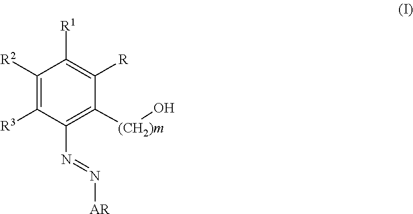

wherein:
R, R¹, R² and R³ are each independently selected from hydrogen, C₁₋₆alkyl, —(CH₂)ₚOR⁴, —C(O)OR⁴, formyl, nitro, cyano, halogen, aryl, substituted aryl, substituted alkyl, —S(O)ₙR⁴, cycloalkyl, —NR⁵R⁶, protected —OH, —CONR⁵R⁶, phosphonic acid, sulfonic acid, phosphinic acid, —SO₂NR⁵R⁶, and a heterocyclic methylene substituent as represented by Formula (III),

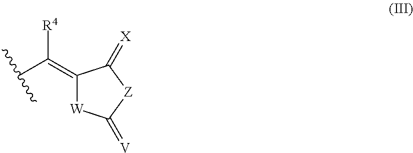

where,
p is 0-6,
n is 0-2,
V, W, X and Z are each independently selected from O, S and NR¹⁶, where R¹⁶ is selected from: hydrogen, alkyl, cycloalkyl, C₁-C₁₂aryl, substituted alkyl, substituted cycloalkyl and substituted C₁-C₁₂aryl,
R⁴ is selected from: hydrogen, alkyl, cycloalkyl, C₁-C₁₂aryl, substituted alkyl, substituted cycloalkyl and substituted C₁-C₁₂aryl, and
R⁵ and R⁶ are each independently selected from hydrogen, alkyl, substituted alkyl, C₃₋₆cycloalkyl, and aryl,
or R⁵ and R⁶ taken together with the nitrogen to which they are attached represent a 5 to 6 member saturated ring containing up to one other heteroatom selected from oxygen and nitrogen;
m is 0-6; and
AR is a cyclic or polycyclic aromatic ring containing from 3 to 16 carbon atoms and optionally containing one or more heteroatoms, provided that when the number of carbon atoms is 3 the aromatic ring contains at least two heteroatoms and when the number of carbon atoms is 4 the aromatic ring contains at least one heteroatom, and optionally substituted with one or more substituents selected from the group consisting of: alkyl, substituted alkyl, aryl, substituted cycloalkyl, substituted aryl, aryloxy, oxo, hydroxy, alkoxy, cycloalkyl, acyloxy, amino, N-acylamino, nitro, cyano, halogen, —C(O)OR⁴, —C(O)NR¹⁰R¹¹, —S(O)₂NR¹⁰R¹¹, —S(O)ₙR⁴ and protected —OH,
   where n is 0-2,
   R⁴ is hydrogen, alkyl, cycloalkyl, C₁-C₁₂aryl, substituted alkyl, substituted cycloalkyl and substituted C₁-C₁₂aryl, and
   R¹⁰ and R¹¹ are independently hydrogen, cycloalkyl, C₁-C₁₂aryl, substituted cycloalkyl, substituted C₁-C₁₂aryl, alkyl or alkyl substituted with one or more substituents selected from the group consisting of: alkoxy, acyloxy, aryloxy, amino, N-acylamino, oxo, hydroxy, —C(O)OR⁴, —S(O)ₙR⁴, —C(O)NR⁴R⁴, —
   S(O)₂NR⁴R⁴, nitro, cyano, cycloalkyl, substituted cycloalkyl, halogen, aryl, substituted aryl and protected —OH,
   or R¹⁰ and R¹¹ taken together with the nitrogen to which they are attached represent a 5 to 6 member saturated ring containing up to one other heteroatom selected from oxygen and nitrogen,
   where R⁴ is as described above and n is 0-2;
and/or pharmaceutically acceptable salts, hydrates, solvates and esters thereof;
provided that at least one of R, R¹, R² and R³ is a substituted aryl group or a heterocyclic methylene substituent as represented in Formula (III).

This invention relates to a method of treating degenerative diseases/injuries, which comprises administering to a subject in need thereof a therapeutically effective amount of a non-peptide TPO receptor agonist of Formula (I).

Included in the present invention are pharmaceutical compositions comprising a pharmaceutical carrier and compounds useful in the methods of the invention.

Also included in the present invention are methods of co-administering non-peptide TPO receptor agonists with further active ingredients.

### DETAILED DESCRIPTION OF THE INVENTION

This invention relates to methods of treating a degenerative disease/injury in a mammal, including a human, in need thereof which comprises administering to such mammal a therapeutically effective amount of a non-peptide TPO receptor agonist, including compounds of Formula (I) as described above.

Included among the compounds that are useful in the present invention are those having Formula (V):

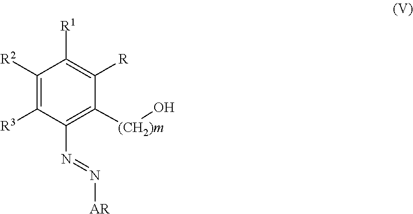

wherein:
R, R¹, R² and R³ are each independently selected from hydrogen, C₁₋₆alkyl, C₁₋₆alkoxy, —(CH₂)ₚOR⁴, —C(O)OR⁴, formyl, nitro, cyano, halogen, aryl, substituted aryl, substituted alkyl, —S(O)ₙR⁴, cycloalkyl, —NR⁵R⁶, protected —OH, —CONR⁵R⁶, phosphonic acid, sulfonic acid, phosphinic acid and —SO₂NR⁵R⁶,
   where,
   p is 0-6,
   n is 0-2,
   R⁴ is selected from: hydrogen, alkyl, cycloalkyl, C₁-C₁₂aryl, substituted alkyl, substituted cycloalkyl and substituted C₁-C₁₂aryl, and
   R⁵ and R⁶ are each independently selected from hydrogen, alkyl, substituted alkyl, C₃₋₆cycloalkyl, and aryl,
   or R⁵ and R⁶ taken together with the nitrogen to which they are attached represent a 5 to 6 member saturated ring containing up to one other heteroatom selected from oxygen and nitrogen;
m is 0-6; and
AR is a cyclic or polycyclic aromatic ring containing from 3 to 16 carbon atoms and optionally containing one or more heteroatoms, provided that when the number of carbon atoms is 3 the aromatic ring contains at least two heteroatoms and when the number of carbon atoms is 4 the aromatic ring contains at least one heteroatom, and optionally substituted with one or more substituents selected from the group consisting of: alkyl, substituted alkyl, aryl, substituted cycloalkyl, substituted aryl, aryloxy, oxo, hydroxy, alkoxy, cycloalkyl, acyloxy, amino, N-acylamino, nitro, cyano, halogen, —C(O)OR⁴, —C(O)NR¹⁰R¹¹, —S(O)₂NR¹⁰R¹¹, —S(O)ₙR⁴ and protected —OH,
   where n is 0-2,
   R⁴ is hydrogen, alkyl, cycloalkyl, C₁-C₁₂aryl, substituted alkyl, substituted cycloalkyl and substituted C₁-C₁₂aryl; and
   R¹⁰ and R¹¹ are independently hydrogen, cycloalkyl, C₁-C₁₂aryl, substituted cycloalkyl, substituted C₁-C₁₂aryl, alkyl or alkyl substituted with one or more substituents selected from the group consisting of: alkoxy, acyloxy, aryloxy, amino, N-acylamino, oxo, hydroxy, —C(O)OR⁴, —S(O)ₙR⁴, —C(O)NR⁴R⁴, —S(O)₂NR⁴R⁴, nitro, cyano, cycloalkyl, substituted cycloalkyl, halogen, aryl, substituted aryl and protected —OH,
   or R¹⁰ and R¹¹ taken together with the nitrogen to which they are attached represent a 5 to 6 member saturated ring containing up to one other heteroatom selected from oxygen and nitrogen,
   where R⁴ is as described above and n is 0-2;
and/or pharmaceutically acceptable salts, hydrates, solvates and esters thereof;
provided that at least one of R, R¹, R² and R³ is a substituted aryl group.

Included among the compounds that are useful in the present invention are those having Formula (II):

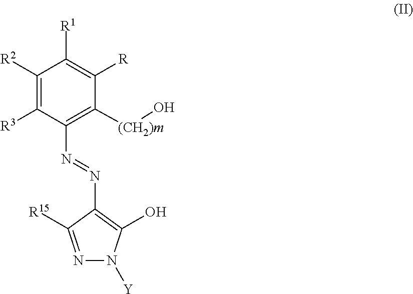

wherein:
R, R¹, R² and R³ are each independently selected from hydrogen, C₁₋₆alkyl, —(CH₂)ₚOR⁴, —C(O)OR⁴, formyl, nitro, cyano, halogen, aryl, substituted aryl, substituted alkyl, —S(O)ₙR⁴, cycloalkyl, —NR⁵R⁶, protected —OH, —CONR⁵R⁶, phosphonic acid, sulfonic acid, phosphinic acid, —SO₂NR⁵R⁶, and a heterocyclic methylene substituent as represented by Formula (III),

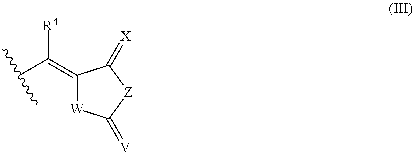

where
p is 0-6,
n is 0-2,
V, W, X and Z are each independently selected from O, S, and NR¹⁶, where R¹⁶ is selected from: hydrogen, alkyl, cycloalkyl, C₁-C₁₂aryl, substituted alkyl, substituted cycloalkyl and substituted C₁-C₁₂aryl,
R⁴ is hydrogen, alkyl, cycloalkyl, C₁-C₁₂aryl, substituted alkyl, substituted cycloalkyl and substituted C₁-C₁₂aryl, and
R⁵ and R⁶ are each independently selected from hydrogen, alkyl, substituted alkyl, C₃₋₆cycloalkyl, and aryl,
or R⁵ and R⁶ taken together with the nitrogen to which they are attached represent a 5 to 6 member saturated ring containing up to one other heteroatom selected from oxygen and nitrogen;
R¹⁵ is selected from the group consisting of alkyl, C₁-C₁₂aryl, hydroxy, alkoxy, substituted alkyl, substituted C₁-C₁₂aryl and halogen;
m is 0-6; and
Y is selected from alkyl, substituted alkyl and a cyclic or polycyclic aromatic ring containing from 3 to 14 carbon atoms and optionally containing from one to three heteroatoms, provided that when the number of carbon atoms is 3 the aromatic ring contains at least two heteroatoms and when the number of carbon atoms is 4 the aromatic ring contains at least one heteroatom, and optionally substituted with one or more substituents selected from the group consisting of: alkyl, substituted alkyl, C₁-C₁₂aryl, substituted cycloalkyl, substituted C₁-C₁₂aryl, hydroxy, aryloxy, alkoxy, cycloalkyl, nitro, cyano, halogen and protected —OH;
and/or pharmaceutically acceptable salts, hydrates, solvates and esters thereof;
provided that at least one of R, R¹, R² and R³ is a substituted aryl group or a heterocyclic methylene substituent as represented in Formula (III).

Included among compounds of Formula (II) that are useful in the current invention are those having Formula (VI):

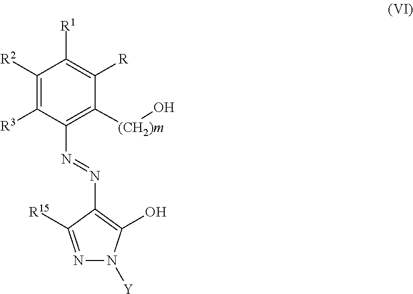

wherein:
R, R¹, R² and R³ are each independently selected from hydrogen, C₁₋₆alkyl, C₁₋₆alkoxy, —(CH₂)ₚOR⁴, —C(O)OR⁴, formyl, nitro, cyano, halogen, aryl, substituted aryl, substituted alkyl, —S(O)ₙR⁴, cycloalkyl, —NR⁵R⁶, protected —OH, —CONR⁵R⁶, phosphonic acid, sulfonic acid, phosphinic acid and —SO₂NR⁵R⁶,
   where
   p is 0-6,
   n is 0-2,
   R⁴ is hydrogen, alkyl, cycloalkyl, C₁-C₁₂aryl, substituted alkyl, substituted cycloalkyl and substituted C₁-C₁₂aryl, and
   R⁵ and R⁶ are each independently selected from hydrogen, alkyl, substituted alkyl, C₃₋₆cycloalkyl, and aryl,
   or R⁵ and R⁶ taken together with the nitrogen to which they are attached represent a 5 to 6 member saturated ring containing up to one other heteroatom selected from oxygen and nitrogen;
R¹⁵ is selected from the group consisting of alkyl, C₁-C₁₂aryl, hydroxy, alkoxy, substituted alkyl, substituted C₁-C₁₂aryl and halogen;
m is 0-6; and
Y is selected from alkyl, substituted alkyl and a cyclic or polycyclic aromatic ring containing from 3 to 14 carbon atoms and optionally containing from one to three heteroatoms, provided that when the number of carbon atoms is 3 the aromatic ring contains at least two heteroatoms and when the number of carbon atoms is 4 the aromatic ring contains at least one heteroatom, and optionally substituted with one or more substituents selected from the group consisting of: alkyl, substituted alkyl, C₁-C₁₂aryl, substituted cycloalkyl, substituted C₁-C₁₂aryl, hydroxy, aryloxy, alkoxy, cycloalkyl, nitro, cyano, halogen and protected —OH;
and pharmaceutically acceptable salts, hydrates, solvates and esters thereof;
provided that at least one of R, R¹, R² and R³ is a substituted aryl group.

Included among the compounds useful in the present invention are those having Formula (VI) in which,

either:
R is a substituted aryl; and R¹ is hydrogen;
   or:
R is hydrogen; and R¹ is a substituted aryl;
   and in either case:
R² and R³ are each independently selected from hydrogen, C₁₋₆alkyl, C₁₋₆alkoxy, nitro, cyano, halogen, aryl, substituted aryl, substituted alkyl, cycloalkyl, phosphonic acid, phosphinic acid and sulfonic acid;
R¹⁵ is selected from the group consisting of alkyl, substituted alkyl, C₁-C₁₂aryl, alkoxy and halogen;
m is 0-4; and
Y is selected from,
   phenyl, pyridinyl and pyrimidinyl, where the phenyl, pyridinyl and pyrimidinyl are optionally substituted with from one to three substituents selected from the group consisting of: alkyl, substituted alkyl, C₁-C₁₂aryl, substituted C₁-C₁₂aryl, alkoxy and halogen;
and pharmaceutically acceptable salts, hydrates, solvates and esters thereof.

Included among the compounds useful in the present invention are those having Formula (VI) in which,
R is a substituted C₁-C₁₂aryl;
   and
R¹ is hydrogen;
R² and R³ are each independently selected from hydrogen, C₁₋₆alkyl, C₁₋₆alkoxy, nitro, cyano, halogen, substituted alkyl and cycloalkyl;
R¹⁵ is selected from the group consisting of alkyl, substituted alkyl, C₁-C₁₂aryl, alkoxy and halogen;
m is 0-2; and
Y is selected from,
   phenyl, pyridinyl and pyrimidinyl, where the phenyl, pyridinyl and pyrimidinyl are optionally substituted with from one to three substituents selected from the group consisting of: alkyl, substituted alkyl, C₁-C₁₂aryl, substituted C₁-C₁₂aryl, alkoxy and halogen;
and pharmaceutically acceptable salts, hydrates, solvates and esters thereof.

Included among the compounds useful in the present invention are those having Formula (VI) in which,
R is a substituted phenyl or pyridinyl ring; and
R¹ is hydrogen;
R² and R³ are each independently selected from hydrogen, C₁₋₆alkyl, substituted alkyl and halogen;
R¹⁵ is selected from the group consisting of C₁₋₄alkyl, C₁₋₄alkoxy, C₁-C₁₂aryl and halogen;
m is 0; and
Y is selected from,
   phenyl, pyridinyl and pyrimidinyl, where the phenyl, pyridinyl and pyrimidinyl is optionally substituted with from one to three substituents selected from the group consisting of: alkyl, substituted alkyl, C₁-C₁₂aryl, substituted C₁-C₁₂aryl, alkoxy and halogen;
and pharmaceutically acceptable salts, hydrates, solvates and esters thereof.

Included among the compounds useful in the present invention are:
4′-{N′-[1-(3,4-Dimethyl phenyl)-3-methyl-5-oxo-1,5-dihydropyrazol-4-ylidene]hydrazino}-3′-hydroxybiphenyl-4-carboxylic acid;
4′-{N′-[1-(3,4-Dimethyl phenyl)-3-methyl-5-oxo-1,5-dihydropyrazol-4-ylidene]hydrazino}-3′-hydroxybiphenyl-3-carboxylic acid;
3′-{N′-[1-(3,4-Dimethyl phenyl)-3-methyl-5-oxo-1,5-dihydropyrazol-4-ylidene]hydrazino}-2′-hydroxybiphenyl-3-carboxylic acid;
3′-{N′-[1-(4-tert-Butylphenyl)-3-methyl-5-oxo-1,5-dihydropyrazol-4-ylidene]hydrazino}-2′-hydroxybiphenyl-3-carboxylic acid;
2-Aza-3′-{N′-[1-(4-tert-butylphenyl)-3-methyl-5-oxo-1,5-dihydropyrazol-4-ylidene]hydrazino}-5′-chloro-2′-hydroxybiphenyl-3-carboxylic acid;
2-Aza-3′-{N′-[1′-(4-tert-butylphenyl)-3-methyl-5-oxo-1,5-dihydropyrazol-4-ylidene]hydrazino}-2′-hydroxybiphenyl-3-carboxylic acid;
3-Aza-3′-{N′-[1-(4-tert-butylphenyl)-3-methyl-5-oxo-1,5-dihydropyrazol-4-ylidene]hydrazino}-2′-hydroxybiphenyl-5-carboxylic acid;
2-Aza-5′-chloro-3′-{N′-[1-(3,4-dimethylphenyl)-3-methyl-5-oxo-1,5-dihydropyrazol-4-ylidene]hydrazino}-2′-hydroxybiphenyl-3-carboxylic acid;
2-Aza-3′-{N′-[1-(4-tert-butylphenyl)-3-methyl-5-oxo-1,5-dihydropyrazol-4-ylidene]hydrazino}-2′-hydroxy-5′-methylbiphenyl-3-carboxylic acid;
2-Aza-3′-{N′-[1-(3,4-dimethylphenyl)-3-methyl-5-oxo-1,5-dihydropyrazol-4-ylidene]hydrazino}-2′-hydroxy-5′-methylbiphenyl-3-carboxylic acid;
3′-{N′-[1-(4-tert-Butylphenyl)-3-methyl-5-oxo-1,5-dihydropyrazol-4-ylidene]hydrazino}-2′-hydroxy-5′-methylbiphenyl-3-carboxylic acid;
3-{N′-[1-(3,4-dimethylphenyl)-3-methyl-5-oxo-1,5-dihydropyrazol-4-ylidene]hydrazino}-2-hydroxy-3′-(tetrazol-5-yl)biphenyl;
3′-{N′-[1-(3,4-dimethylphenyl)-3-methyl-5-oxo-1,5-dihydropyrazol-4-ylidene]hydrazino}-5′-fluoro-2′-hydroxybiphenyl-3-carboxylic acid;
7-({N′-[1-(3,4-dimethylphenyl)-3-methyl-5-oxo-1,5-dihydropyrazol-4-ylidene]hydrazino}-2-hydroxyphenyl)quinolin-4[1H]-one-3-carboxylic acid;
7-({N′-[1-(4-tert-butylphenyl)-3-methyl-5-oxo-1,5-dihydropyrazol-4-ylidene]hydrazino}-2-hydroxyphenyl)quinolin-4[1H]-one-3-carboxylic acid;
3-Aza-3′-{N′-[1-(3,4-dimethylphenyl)-3-methyl-5-oxo-1,5-dihydropyrazol-4-ylidene]hydrazino}-2′-hydroxybiphenyl-5-carboxylic acid;
3-Aza-3′-(N′-[1-{3-methyl-[4-(1-methylethyl)phenyl]-5-oxo-1,5-dihydropyrazol-4-ylidene}hydrazino)-2′-hydroxybiphenyl-5-carboxylic acid;
3-Aza-3′-{N′-[1-(4-tertbutylphenyl-3-methyl-5-oxo-1,5-dihydropyrazol-4-ylidene]hydrazino}-2′-hydroxybiphenyl-5-carboxylic acid;
5′-Chloro-3′-{N′-[1-(3,4-dimethylphenyl)-3-methyl-5-oxo-1,5-dihydropyrazol-4-ylidene]hydrazino}-2′-hydroxybiphenyl-3-carboxylic acid;
3′-{N′-[1-(3,4-Dimethylphenyl)-3,5-dioxo-1,5-dihydropyrazol-4-ylidene]hydrazino}-2′-hydroxybiphenyl-3-carboxylic acid;
3′-{N′-[1-(2-Ethoxy-2-oxoethyl)-3-methyl-5-oxo-1,5-dihydropyrazol-4-ylidene]hydrazino}-2′-hydroxybiphenyl-3-carboxylic acid;
3-{N′-[1-(3,4-dimethylphenyl)-3-methyl-5-oxo-1,5-dihydropyrazol-4-ylidene]hydrazino}-2-hydroxy-4′-(tetrazol-5-yl)biphenyl;
3′-(N′-{1-[2-(N-tert-butyl)amino-2-oxoethyl]-3-methyl-5-oxo-1,5-dihydropyrazol-4-ylidene}hydrazino)-2′-hydroxybiphenyl-3-carboxylic acid;
3′-{N′-[3-Chloro-1-(3,4-dimethylphenyl)-5-oxo-1,5-dihydropyrazol-4-ylidene]hydrazino}-2′-hydroxybiphenyl-3-carboxylic acid;
5-chloro-3-{N′-[1-(3,4-dimethylphenyl)-3-methyl-5-oxo-1,5-dihydropyrazol-4-ylidene]hydrazino}-2-hydroxy-4′-(tetrazol-5-yl)biphenyl;
3′-{N′-[1-(3,4-Dimethylphenyl)-3-methyl-5-oxo-1,5-dihydropyrazol-4-ylidene]hydrazino}-2′-hydroxybiphenyl-3,5-dicarboxylic acid;
3-Aza-3′-{N′-[1-(3,4-dimethylphenyl)-3-methyl-5-oxo-1,5-dihydropyrazol-4-ylidene]hydrazino}-2′-hydroxy-5′-methylbiphenyl-5-carboxylic acid;
3′-{N′-[1-(3,4-Dimethylphenyl)-3-methyl-5-oxo-1,5-dihydropyrazol-4-ylidene]hydrazino}-2′-hydroxybiphenyl-4-carboxylic acid;
3′-{N′-[1-(3,4-Dimethylphenyl)-3-methoxy-5-oxo-1,5-dihydropyrazol-4-ylidene]hydrazino}-2′-hydroxybiphenyl-3-carboxylic acid;
3′-{N′-[1-(4-methoxyphenyl)-3-methyl-5-oxo-1,5-dihydropyrazol-4-ylidene]hydrazino}-2′-hydroxybiphenyl-3-carboxylic acid;
(3-{N′-[1-(3,4-dimethyl phenyl)-3-methyl-5-oxo-1,5-dihydropyrazol-4-ylidene]hydrazino}-2-hydroxy-3′-biphenyl)-1,1,1,-trifluoromethanesulfonamide;
3′-{N′-[1-(3,4-Dichlorophenyl)-3-methyl-5-oxo-1,5-dihydropyrazol-4-ylidene]hydrazino}-2′-hydroxybiphenyl-3-carboxylic acid;
3′-{N′-[3-methyl-5-oxo-1-(3-trifluoromethylphenyl)-1,5-dihydropyrazol-4-ylidene]hydrazino}-2′-hydroxybiphenyl-3-carboxylic acid;
8-{N′-[1-(3,4-dimethylphenyl)-3-methyl-5-oxo-1,5-dihydropyrazol-4-ylidene]hydrazino}quinolin-4[1H]-one-3-carboxylic acid;
3′-{N′-[3-methyl-5-oxo-1-(4-trifluoromethylphenyl)-1,5-dihydropyrazol-4-ylidene]hydrazino}-2′-hydroxybiphenyl-3-carboxylic acid;
3′-{N′-[3-methyl-5-oxo-1-(4-N-methylcarboxamidolphenyl)-1,5-dihydropyrazol-4-ylidene]hydrazino}-2′-hydroxybiphenyl-3-carboxylic acid;
N-[1-(3′-{N′[1-(3,4-dimethylphenyl)-3-methyl-5-oxo-1,5-dihydropyrazol-4-ylidene]hydrazino}-2′-hydroxybiphenyl-3-yl)methanoyl]methanesulfonamide;
3′-{N′-[3-methyl-5-oxo-1-phenyl-1,5-dihydropyrazol-4-ylidene]hydrazino}-2′-hydroxybiphenyl-3-carboxylic acid;
3′-{N′-[3-methyl-1-(4-methylphenyl)-5-oxo-1,5-dihydropyrazol-4-ylidene]hydrazino}-2′-hydroxybiphenyl-3-carboxylic acid;
3′-{N′-[1-(4-chlorophenyl)-3-methyl-5-oxo-1,5-dihydropyrazol-4-ylidene]hydrazino}-2′-hydroxybiphenyl-3-carboxylic acid;
3′-{N′-[1-(4-fluorophenyl)-3-methyl-5-oxo-1,5-dihydropyrazol-4-ylidene]hydrazino}-2′-hydroxybiphenyl-3-carboxylic acid;
3′-{N′-[3-methyl-5-oxo-1-(4-trifluoromethoxyphenyl)-1,5-dihydropyrazol-4-ylidene]hydrazino}-2′-hydroxybiphenyl-3-carboxylic acid;
3′-{N′-[1-(3,4-dimethylphenyl)-3-ethoxy-5-oxo-1,5-dihydropyrazol-4-ylidene]hydrazino}-2′-hydroxybiphenyl-3-carboxylic acid;
3′-{N′-[1-(3,4-dimethylphenyl)-3-(1-methylethoxy)-5-oxo-1,5-dihydropyrazol-4-ylidene]hydrazino}-2′-hydroxybiphenyl-3-carboxylic acid;
3′-{N′-[3-tert-butyl-1-(3,4-dimethylphenyl)-5-oxo-1,5-dihydropyrazol-4-ylidene]hydrazino}-2′-hydroxybiphenyl-3-carboxylic acid;
3′-{N′-[3-methyl-1-(4-methyl-2,3,5,6-tetrafluorophenyl)-5-oxo-1,5-dihydropyrazol-4-ylidene]hydrazino}-2′-hydroxybiphenyl-3-carboxylic acid;
3′-{N′-[1-(4-fluoro-3-methylphenyl)-3-methyl-5-oxo-1,5-dihydropyrazol-4-ylidene]hydrazino}-2′-hydroxybiphenyl-3-carboxylic acid;
3′-{N′-[1-(3.4-dimethylphenyl)-3-phenyl-5-oxo-1,5-dihydropyrazol-4-ylidene]hydrazino}-2′-hydroxybiphenyl-3-carboxylic acid;
3-{N′-[1-(3,4-dimethylphenyl)-5-oxo-3-phenyl-1,5-dihydropyrazol-4-ylidene]hydrazino}-2-hydroxy-3′-tetrazol-5-ylbiphenyl;
3-{N′-[1-(3,4-dimethylphenyl)-3-methoxy-5-oxo-1,5-dihydropyrazol-4-ylidene]hydrazino}-2-hydroxy-3′-tetrazol-5-ylbiphenyl;
3-{N′-[1-(3,4-dimethylphenyl)-3-ethoxy-5-oxo-1,5-dihydropyrazol-4-ylidene]hydrazino}-2-hydroxy-3′-tetrazol-5-ylbiphenyl;
3-{N′-[1-(3,4-dimethylphenyl)-3-(1-methylethoxy)-5-oxo-1,5-dihydropyrazol-4-ylidene]hydrazino}-2-hydroxy-3′-tetrazol-5-ylbiphenyl;
3-{N′-[1-(4-fluorophenyl)-3-methyl-5-oxo-1,5-dihydropyrazol-4-ylidene]hydrazino}-2-hydroxy-3′-tetrazol-5-ylbiphenyl;
3-{N′-[1-(4-fluoro-3-methylphenyl)-3-methyl-5-oxo-1,5-dihydropyrazol-4-ylidene]hydrazino}-2-hydroxy-3′-tetrazol-5-ylbiphenyl;
3-{N′-[3-methyl-5-oxo-1-(4-trifluoromethylphenyl)-1,5-dihydropyrazol-4-ylidene]hydrazino}-2-hydroxy-3′-tetrazol-5-ylbiphenyl;
3′-{N′-[1-(3.4-dimethylphenyl)-3-(pyridin-4-yl-5-oxo-1,5-dihydropyrazol-4-ylidene]hydrazino}-2′-hydroxybiphenyl-3-carboxylic acid;
3-{N′-[1-(3,4-dimethylphenyl)-3-pyridin-4-yl-5-oxo-1,5-dihydropyrazol-4-ylidene]hydrazino}-2-hydroxy-3′-tetrazol-5-ylbiphenyl;
3-{N′-[1-(3,4-dimethylphenyl)-3-pyridin-2-yl-5-oxo-1,5-dihydropyrazol-4-ylidene]hydrazino}-2-hydroxy-3′-tetrazol-5-ylbiphenyl;
3′-{N′-[1-(3.4-dimethylphenyl)-3-(pyridin-2-yl-5-oxo-1,5-dihydropyrazol-4-ylidene]hydrazino}-2′-hydroxybiphenyl-3-carboxylic acid;
3-{N′-[1-(3-fluoro-4-methylphenyl)-3-methyl-5-oxo-1,5-dihydropyrazol-4-ylidene]hydrazino}-2-hydroxy-3′-tetrazol-5-ylbiphenyl;
3′-{N′-[1-(3-fluoro-4-methylphenyl)-3-methyl-5-oxo-1,5-dihydropyrazol-4-ylidene]hydrazino}-2′-hydroxybiphenyl-3-carboxylic acid;
3′-{N′[3-methyl-5-oxo-1-(4-trifluoromethylpyrimidin-2-yl)-1,5-dihydropyrazol-4-ylidene]hydrazino}-2′-hydroxybiphenyl-3-carboxylic acid;
3′-N-tert-butoxycarbonylamino-3-{N′-[1-(3,4-Dimethylphenyl)-3-methyl-5-oxo-1,5-dihydropyrazol-4-ylidene]hydrazino}-2-hydroxybiphenyl;
3′-amino-3-{N′-[1-(3,4-dimethylphenyl)-3-methyl-5-oxo-1,5-dihydropyrazol-4-ylidene]hydrazino}-2-hydroxybiphenyl;
3-{N′-[1-(3-fluorophenyl)-3-methyl-5-oxo-1,5-dihydropyrazol-4-ylidene]hydrazino}-2-hydroxy-3′-tetrazol-5-ylbiphenyl;
3′-{N′-[1-(3-fluorophenyl)-3-methyl-5-oxo-1,5-dihydropyrazol-4-ylidene]hydrazino}-2′-hydroxybiphenyl-3-carboxylic acid;
3-{N′-[3-methyl-5-oxo-1-(2,3,4,5,6-pentafluorophenyl)-1,5-dihydropyrazol-4-ylidene]hydrazino}-2-hydroxy-3′-tetrazol-5-ylbiphenyl;
3′-{N′[3-methyl-5-oxo-1-(2,3,4,5,6-pentafluorophenyl)-1,5-dihydropyrazol-4-ylidene]hydrazino}-2′-hydroxybiphenyl-3-carboxylic acid;
3′-{N′-[1-(3,4-difluorophenyl)-3-methyl-5-oxo-1,5-dihydropyrazol-4-ylidene]hydrazino}-2′-hydroxybiphenyl-3-carboxylic acid;
3′-{N′-[1-(3,4-dimethylphenyl)-3-methoxymethyl-5-oxo-1,5-dihydropyrazol-4-ylidene]hydrazino}-2′-hydroxybiphenyl-3-carboxylic acid;
3-{N′-[1-(3,4-dimethylphenyl)-3-methoxymethyl-5-oxo-1,5-dihydropyrazol-4-ylidene]hydrazino}-2-hydroxy-3′-tetrazol-5-ylbiphenyl;
3-{N′-[1-(3,4-difluorophenyl)-3-methyl-5-oxo-1,5-dihydropyrazol-4-ylidene]hydrazino}-2-hydroxy-3′-tetrazol-5-ylbiphenyl;
3′-{N′-[1-(3,4-dimethylphenyl)-5-oxo-3-trifluoromethyl-1,5-dihydropyrazol-4-ylidene]hydrazino}-2′-hydroxybiphenyl-3-carboxylic acid;
3′-{N′-[1-(3,4-dimethylphenyl)-3-methyl-5-oxo-1,5-dihydropyrazol-4-ylidene]hydrazino}-6-fluoro-2′-hydroxybiphenyl-3-carboxylic acid;
3′-{N′-[1-(3,4-dimethylphenyl)-5-oxo-3-propyl-1,5-dihydropyrazol-4-ylidene]hydrazino}-2′-hydroxybiphenyl-3-carboxylic acid;
3-{N′-[1-(3,4-dimethylphenyl)-5-oxo-3-propyl-1,5-dihydropyrazol-4-ylidene]hydrazino}-2-hydroxy-3′-tetrazol-5-ylbiphenyl;
3′-{N′-[1-(3,4-dimethylphenyl)-3-(1-methyl-1H-pyrrol-3-yl)-5-oxo-1,5-dihydropyrazol-4-ylidene]hydrazino}-2′-hydroxybiphenyl-3-carboxylic acid;
{N′-[1-(3,4-dimethyl phenyl)-3-(1-methyl-1H-pyrrol-3-yl)-5-oxo-1,5-dihydropyrazol-4-ylidene]hydrazino}-2-hydroxy-3′-tetrazol-5-ylbiphenyl;
3′-{N′-[1-(3,4-dimethylphenyl)-3-furan-2-yl-5-oxo-1,5-dihydropyrazol-4-ylidene]hydrazino}-2′-hydroxybiphenyl-3-carboxylic acid;
3-{N′-[1-(3,4-dimethylphenyl)-3-furan-2-yl-5-oxo-1,5-dihydropyrazol-4-ylidene]hydrazino}-2-hydroxy-3′-tetrazol-5-ylbiphenyl;
N-(2′-hydroxy-3′-{N′[3-methyl-5-oxo-1-(4-trifluoromethyl-phenyl)-1,5-dihydro-pyrazol-4-ylidene]hydrazino}biphenyl-3-yl)-1,1,1-trifluoromethanesulfonamide;
N-(2′-hydroxy-3′-{N′-[1-(3-fluoro-4-methyl phenyl)-3-methyl-5-oxo-1,5-dihydro-pyrazol-4-ylidene]hydrazino}biphenyl-3-yl)-1,1,1-trifluoromethanesulfonamide;
N-(2′-hydroxy-3′-{N′-[1-(4-fluoro-3-methylphenyl)-3-methyl-5-oxo-1,5-dihydro-pyrazol-4-ylidene]hydrazino}biphenyl-3-yl)-1,1,1-trifluoromethanesulfonamide;
N-(2′-hydroxy-3′-{N′-[1-(3,4-difluorophenyl)-3-methyl-5-oxo-1,5-dihydro-pyrazol-4-ylidene]hydrazino}biphenyl-3-yl)-1,1,1-trifluoromethanesulfonamide;
N-(3′-{N′-[1-(3,4-dimethyl phenyl)-3-methyl-5-oxo-1,5-dihydropyrazol-4-ylidene]hydrazino}-2′-hydroxybiphenyl-3-yl)guanidine;
3′-{N′-[1-(3,4-dimethylphenyl)-3-ethyl-5-oxo-1,5-dihydropyrazol-4-ylidene]hydrazino}-2′-hydroxybiphenyl-3-carboxylic acid;
3-{N′-[1-(3,4-dimethylphenyl)-3-ethyl-5-oxo-1,5-dihydropyrazol-4-ylidene]hydrazino}-2-hydroxy-3′-tetrazol-5-ylbiphenyl;
3′-{N′-[1-(3,4-dimethylphenyl)-5-oxo-3-thien-2-yl-1,5-dihydropyrazol-4-ylidene]hydrazino}-2′-hydroxybiphenyl-3-carboxylic acid;
3′-{N′-[3-cyclopropyl-1-(3,4-dimethylphenyl)-5-oxo-1,5-dihydropyrazol-4-ylidene]hydrazino}-2′-hydroxybiphenyl-3-carboxylic acid;
3′-{N′-[1-(3,4-dimethylphenyl)-5-oxo-3-thiazol-2-yl-1,5-dihydropyrazol-4-ylidene]hydrazino}-2′-hydroxybiphenyl-3-carboxylic acid;
3′-{N′-[1-(3,4-dimethylphenyl)-5-oxo-1,5-dihydropyrazol-4-ylidene]hydrazino}-2′-hydroxybiphenyl-3-carboxylic acid;
3′-{N′-[1-(3,4-dimethylphenyl)-3-(1-methylethyl)-5-oxo-1,5-dihydropyrazol-4-ylidene]hydrazino}-2′-hydroxybiphenyl-3-carboxylic acid;
3′-{N′-[3-(benzyloxymethyl)-1-(3,4-dimethyl phenyl)-5-oxo-1,5-dihydropyrazol-4-ylidene]hydrazino}-2′-hydroxybiphenyl-3-carboxylic acid;
3′-{N′-[3-ethyl-5-oxo-1-(4-trifluoromethylphenyl)-1,5-dihydropyrazol-4-ylidene]hydrazino}-2′-hydroxybiphenyl-3-carboxylic acid;
3′-{N′-[5-oxo-1-(4-trifluoromethylphenyl)-1,5-dihydropyrazol-4-ylidene]hydrazino}-2′-hydroxybiphenyl-3-carboxylic acid;
3′-{N′-[-1-(3,4-dimethylphenyl)-3-hydroxymethyl-5-oxo-1,5-dihydropyrazol-4-ylidene]hydrazino}-2′-hydroxybiphenyl-3-carboxylic acid;
3′-{N′-[3-benzyloxymethyl-5-oxo-1-(4-trifluoromethylphenyl)-1,5-dihydropyrazol-4-ylidene]hydrazino}-2′-hydroxybiphenyl-3-carboxylic acid;
3′-{N′-[-1-(3,4-dimethylphenyl)-3-methylsulfanylmethyl-5-oxo-1,5-dihydropyrazol-4-ylidene]hydrazino}-2′-hydroxybiphenyl-3-carboxylic acid;
3′-{N′-[-1-(3,4-dimethylphenyl)-5-oxo-3-thiophen-3-yl-1,5-dihydropyrazol-4-ylidene]hydrazino}-2′-hydroxybiphenyl-3-carboxylic acid;
3′-{N′-[5-oxo-1-(4-trifluoromethylphenyl)-3-thiophen-3-yl-1,5-dihydropyrazol-4-ylidene]hydrazino}-2′-hydroxybiphenyl-3-carboxylic acid;
3′-{N′-[5-oxo-1-(4-trifluoromethylphenyl)-3-methylsulfanylmethyl-1,5-dihydropyrazol-4-ylidene]hydrazino}-2′-hydroxybiphenyl-3-carboxylic acid;
N-(3′-{N′-[1-(3,4-dimethylphenyl)-3-methyl-5-oxo-1,5-dihydro-pyrazol-4-ylidene]hydrazino}-2′-hydroxybiphenyl-3-yl)methanesulfonamide;
3′-[N′-(1-benzo[1,3]dioxol-5-yl-3-methyl-5-oxo-1,5-dihydropyrazol-4-ylidene)hydrazino]-2′-hydroxybiphenyl-3-carboxylic acid;
3′-{N′-[1-(3,5-dimethylphenyl)-3-methyl-5-oxo-1,5-dihydropyrazol-4-ylidene]hydrazino}-2′-hydroxybiphenyl-3-carboxylic acid;
3′-{N′-[1-(3,4-dimethylphenyl)-3-methyl-5-oxo-1,5-dihydropyrazol-4-ylidene]hydrazino}-4′-hydroxybiphenyl-4-carboxylic acid;
3′-{N′-[1-(3-chloro-4-methylphenyl)-3-methyl-5-oxo-1,5-dihydropyrazol-4-ylidene]hydrazino}-2′-hydroxybiphenyl-3-carboxylic acid;
3′-{N′-[1-(3,4-dimethylphenyl)-3-methyl-5-oxo-1,5-dihydropyrazol-4-ylidene]hydrazino}-4′-hydroxybiphenyl-3-carboxylic acid;
3′-{N′-[1-(3,4-dimethylphenyl)-3-methyl-5-oxo-1,5-dihydropyrazol-4-ylidene]hydrazino}-2′-hydroxybiphenyl-3-phosphonic acid;
3′-{N′-[1-(3,4-dimethylphenyl)-3-methyl-5-oxo-1,5-dihydropyrazol-4-ylidene]hydrazino}-2′-hydroxybiphenyl-3,4-dicarboxylic acid;
2′,6-dihydroxy-3′-{N′-[1-(3,4-dimethylphenyl)-3-methyl-5-oxo-1,5-dihydropyrazol-4-ylidene]hydrazino}biphenyl-3-carboxylic acid;
4-aza-3′-{N′-[1-(3,4-dimethylphenyl)-3-methyl-5-oxo-1,5-dihydropyrazol-4-ylidene]hydrazino}-2′-hydroxybiphenyl-5-carboxylic acid;
3′-{N′-[1-(3,4-dimethylphenyl)-5-oxo-1,5-dihydropyrazol-4-ylidene]hydrazino}-2′-hydroxybiphenyl-3-carboxylic acid;
3′-{N′-[1-(3,4-dimethylphenyl)-3-methyl-5-oxo-1,5-dihydropyrazol-4-ylidene]hydrazino}-2′-hydroxybiphenyl-3-sulfonic acid; and
5-(3′-{N′-[1-(3,4-Dimethylphenyl)-3-methyl-5-oxo-1,5-dihydropyrazol-4-ylidene]hydrazino}-2′-hydroxybiphenyl-3-ylmethylene)thiazolidine-2,4-dione;
   and/or pharmaceutically acceptable salts, hydrates, solvates and esters thereof.

Included among the non-peptide TPO receptor agonists of the invention are the non-peptide compounds described in:
WO 02/59099;
WO 02/59100;
EP 1 207 155;
EP 1 253 142A1;
WO 01/92211 A1;
WO 01/53267-A1;
EP 1 104 674-A1; and
WO 01/07423-A1.

Included among the compounds of the above listed applications that are useful in the present invention are:
N-[4-(5-bromo-2-thienyl)-1,3-thiazol-2-yl]-4-[(Z)-(2,4-dioxo-1,3-thiazolidin-5-ylidene)methyl]benzamide;
N-[4-(3,4-dimethylphenyl)-1,3-thiazol-2-yl]-4-[(Z)-(2,4-dioxo-1,3-thiazolidin-5-ylidene)methyl]benzamide;
N-{4-[4-(1,1-dimethylethyl)phenyl]-1,3-thiazol-2-yl}-4-[(Z)-(2,4-dioxo-1,3-thiazolidin-5-ylidene)methyl]benzamide;
N-[4-(3,4-dichlorophenyl)-1,3-thiazol-2-yl]-4-[(Z)-(2,4-dioxo-1,3-thiazolidin-5-ylidene)methyl]benzamide; and
(2E)-3-[4-({[4-(3,4-dichlorophenyl)-1,3-thiazol-2-yl]amino}carbonyl)phenyl]-2-methyl-2-propenoic acid;
   and/or pharmaceutically acceptable salts, hydrates, solvates and esters thereof.

Included among the non-peptide TPO receptor agonists of the invention are the non-peptide compounds described in:
WO 99/11262.

Non-peptide TPO receptor agonists are included in the pharmaceutical compositions of the invention and used in the methods of the invention.

By the term “protected hydroxy” or “protected —OH” as used herein, is meant the alcoholic or carboxylic-OH groups which can be protected by conventional blocking groups in the art such as described in “Protective Groups In Organic Synthesis” by Theodora W. Greene, Wiley-Interscience, 1981, New York. Compounds containing protected hydroxy groups may also be useful as intermediates in the preparation of the pharmaceutically active compounds of the invention.

By the term “aryl” as used herein, unless otherwise defined, is meant a cyclic or polycyclic aromatic ring containing from 1 to 14 carbon atoms and optionally containing from one to five heteroatoms, provided that when the number of carbon atoms is 1 the aromatic ring contains at least four heteroatoms, when the number of carbon atoms is 2 the aromatic ring contains at least three heteroatoms, when the number of carbons is 3 the aromatic ring contains at least two heteroatoms and when the number of carbon atoms is 4 the aromatic ring contains at least one heteroatom.

By the term “C₁-C₁₂aryl” as used herein, unless otherwise defined, is meant phenyl, naphthalene, 3,4-methylenedioxyphenyl, pyridine, biphenyl, quinoline, pyrimidine, quinazoline, thiophene, furan, pyrrole, pyrazole, imidazole and tetrazole.

When referring to compounds of Formula (I) and (II), the term “substituted” as used herein, unless otherwise defined, is meant that the subject chemical moiety has one or more substituents selected from the group consisting of: —CO₂R²⁰, aryl, —C(O)NHS(O)₂R²⁰, —NHS(O)₂R²⁰, hydroxyalkyl, alkoxy, —C(O)NR²¹R²², acyloxy, alkyl, amino, N-acylamino, hydroxy, —(CH₂)_{g}C(O)OR⁸, —S(O)ₙR⁸, nitro, tetrazole, cyano, oxo, halogen, trifluoromethyl, protected —OH and a heterocyclic methylene substituent as represented by Formula (III),

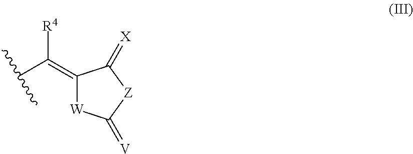

where g is 0-6; R⁸ is hydrogen or alkyl; R²⁰ is selected form hydrogen, C₁-C₄alkyl, aryl and trifluoromethyl; R²¹ and R²² are independently selected form hydrogen, C₁-C₄alkyl, aryl and trifluoromethyl; V, W, X and Z are each independently selected from O, S, and NR¹⁶, where R¹⁶ is selected from: hydrogen, alkyl, cycloalkyl, C₁-C₁₂aryl, substituted alkyl, substituted cycloalkyl and substituted C₁-C₁₂aryl; and n is 0-2.

When referring to compounds of Formula (V) and (VI), the term “substituted” as used herein, unless otherwise defined, is meant that the subject chemical moiety has one or more substituents selected from the group consisting of: —CO₂R²⁰, aryl, —C(O)NHS(O)₂R²⁰, —NHS(O)₂R²⁰, hydroxyalkyl, alkoxy, —C(O)NR²¹R²², acyloxy, alkyl, amino, N-acylamino, hydroxy, —(CH₂)_{g}C(O)OR⁸, —S(O)ₙR⁸, nitro, tetrazole, cyano, oxo, halogen, trifluoromethyl and protected —OH, where g is 0-6, R⁸ is hydrogen or alkyl, R²⁰ is selected form hydrogen, C₁-C₄alkyl, aryl and trifluoromethyl, and R²¹ and R²² are independently selected form hydrogen, C₁-C₄alkyl, aryl and trifluoromethyl, and n is 0-2.

By the term “alkoxy” as used herein is meant —Oalkyl where alkyl is as described herein including —OCH₃ and —OC(CH₃)₂CH₃.

The term “cycloalkyl” as used herein unless otherwise defined, is meant a nonaromatic, unsaturated or saturated, cyclic or polycyclic C₃-C₁₂.

Examples of cycloalkyl and substituted cycloalkyl substituents as used herein include: cyclohexyl, 4-hydroxy-cyclohexyl, 2-ethylcyclohexyl, propyl 4-methoxycyclohexyl, 4-methoxycyclohexyl, 4-carboxycyclohexyl, cyclopropyl and cyclopentyl.

By the term “acyloxy” as used herein is meant —OC(O)alkyl where alkyl is as described herein. Examples of acyloxy substituents as used herein include: —OC(O)CH₃, —OC(O)CH(CH₃)₂ and —OC(O)(CH₂)₃CH₃.

By the term “N-acylamino” as used herein is meant —N(H)C(O)alkyl, where alkyl is as described herein. Examples of N-acylamino substituents as used herein include: —N(H)C(O)CH₃, —N(H)C(O)CH(CH₃)₂ and —N(H)C(O)(CH₂)₃CH₃.

By the term “aryloxy” as used herein is meant —Oaryl where aryl is phenyl, naphthyl, 3,4-methylenedioxyphenyl, pyridyl or biphenyl optionally substituted with one or more substituents selected from the group consisting of: alkyl, hydroxyalkyl, alkoxy, trifluoromethyl, acyloxy, amino, N-acylamino, hydroxy, —(CH₂)_{g}C(O)OR⁸, —S(O)ₙR⁸, nitro, cyano, halogen and protected —OH, where g is 0-6, R⁸ is hydrogen or alkyl, and n is 0-2. Examples of aryloxy substituents as used herein include: phenoxy, 4-fluorophenyloxy and biphenyloxy.

By the term “heteroatom” as used herein is meant oxygen, nitrogen or sulfur.

By the term “halogen” as used herein is meant a substituent selected from bromide, iodide, chloride and fluoride.

By the term “alkyl” and derivatives thereof and in all carbon chains as used herein is meant a linear or branched, saturated or unsaturated hydrocarbon chain, and unless otherwise defined, the carbon chain will contain from 1 to 12 carbon atoms. Examples of alkyl substituents as used herein include: —CH₃, —CH₂—CH₃, —CH₂—CH₂—CH₃, —CH(CH₃)₂, —C(CH₃)₃, —(CH₂)₃—CH₃, —CH₂—CH(CH₃)₂, —CH(CH₃)—CH₂—CH₃, —CH═CH₂, and —C≡C—CH₃.

By the term “treating” and derivatives thereof as used herein, is meant prophylactic and therapeutic therapy. Prophylactic therapy is appropriate, for example, when a subject is considered at high risk for developing a degenerative disease/injury, such as prior to heart surgery or prior to the administration a pharmaceutically active compound that is known to cause injury to cardiovascular tissue.

By the phrases “to a therapeutic extent” and “therapeutically effective amount” and derivatives thereof as used herein, unless otherwise defined, is meant that the incidence of degenerative disease/injury in patients treated with a non-peptide TPO receptor agonist is prevented or reduced in severity in comparison to untreated patients. For example, when administering a non-peptide TPO receptor agonist of the invention in the treatment of cardiovascular disease (for example any cardiovascular injury or, more specifically, myocardial infarction) a “therapeutic extent” or “therapeutically effective amount” would be to the extent or the amount that would increase survival post cardiovascular event, e.g. myocardial infarction. For example, when administering a non-peptide TPO receptor agonist of the invention in the treatment of cardiovascular disease (for example any cardiovascular injury or, more specifically, myocardial infarction) a “therapeutic extent” or “therapeutically effective amount” would be an improved electrocardiogram (EKG) in patients treated with a non-peptide TPO receptor agonist of the invention when compared to untreated patients.

By the phrase “non-peptide” as used herein is meant a chemical compound, or a protein or peptide not comprised primarily of natural amino acids. Suitably, the “non-peptide” is a small molecule chemical compound having a molecular weight under 1,500 daltons, suitably under 1,000 daltons.

By the term “primarily” as used above is meant about 60% by weight of naturally occurring amino acid residue.

By the phrase “degenerative diseases/injuries” and derivatives thereof as used herein, unless otherwise defined, is meant: nervous system disorders, including transverse myelitis, multiple sclerosis, demyelination occurring after trauma to the brain or spinal cord, acute brain injury, head trauma, spinal cord injury, peripheral nerve injury, ischaemic brain injury, hereditary myelin disorder of the CNS, epilepsy, perinatal asphxia, asphyxia, anoxia, status epilepticus, and stroke; baldness, such as male pattern baldness and alopecia greata; neurodegenerative diseases, such as Alzheimer's disease, Parkinson disease, Huntington's disease, and amyotrophic lateral sclerosis; tissue reparation disorders, including cardiovascular disorders, myocardial infarction, cardiovascular disease, gastrointestinal disease, kidney disease and liver disease; damaged tissue, such as flesh wounds, age damaged cells and age damaged tissue; lupus; and diabetes/diabetes mellitus.

As used herein stroke refers to a Cerebral Vascular Incident and includes acute thromboembolic stroke. The term stroke, as used herein, also includes both focal and global ischemia. Also included in stroke, as used herein, are transient cerebral ischemic attacks and other cerebral vascular problems accompanied by cerebral ischemia. A patient undergoing carotid endarterectomy specifically or other cerebrovascular or vascular surgical procedures in general, or diagnostic vascular procedures including cerebral angiography and the like, are also examples of stroke, as used herein.

Injuries that are included within the term “degenerative diseases/injuries” are: head trauma, spinal cord trauma and injury from general anoxia, hypoxia, hypoglycemia, hypotension, as well as similar injuries seen during procedures from embole, hyperfusion, and hypoxia.

Further injuries treatable by the present invention include those which occur, during cardiac bypass surgery, in incidents of intracranial hemorrhage, in perinatal asphyxia, in cardiac arrest, and status epilepticus.

Additional degenerative diseases treatable by the present invention are disease states caused by excessive bone loss or cartilage or matrix degradation such as: osteoporosis, glucocorticoid induced osteoporosis, Paget's disease, abnormally increased bone turnover, periodontal disease, gingivitis, tooth loss, bone fractures, arthritis, rheumatoid arthritis, osteoarthritis, periprosthetic osteolysis, osteogenesis imperfecta, or metastatic bone disease. It is part of the present invention that treatment with a non-peptide TPO receptor agonist, as described herein, is useful in reducing the risk of bone fractures and in increasing bone mineral density.

Additional degenerative diseases treatable by the present invention are degenerative diseases of the eye such as: macular degeneration, dry eye syndrome, cataracts, diabetic retinopathy, glaucoma, vitreous disease and retinal degeneration.

An additional degenerative disease treatable by the present invention is AIDS.

Because the in vivo administration of the non-peptide TPO receptor agonist of the present invention, in mammals, including humans, exhibits therapeutic activity in diseases/injuries that are therapeutically treatable by stem cells/stem cell therapy, the non-peptide TPO receptor agonist of the present invention are useful in treating diseases/injuries that are known to be treatable by stem cells/stem cell therapy or found to be treatable by stem cells/stem cell therapy.

An example of damaged tissue, such as flesh wounds, as used herein is vascular access dysfunction in mammals, including humans. Suitably, the vascular access dysfunction is in association with the insertion, maintenance or repair of an indwelling shunt, fistula or catheter, suitably a large bore catheter, into a vein.

Further, vascular access dysfunction in chemotherapy patients is generally caused by outflow stenoses in the venous circulation and results in a decreased ability to administer medications to cancer patients. Often the outflow stenoses is so severe as to require intervention.

Additionally, vascular access dysfunction in total parenteral nutrition (TPN) patients is generally caused by outflow stenoses in the venous circulation and results in reduced ability to care for these patients.

The current invention is directed to the prevention or reduction of vascular access dysfunction in association with the insertion or repair of an indwelling shunt, fistula or catheter, suitably a large bore catheter, into a vein in a mammal, particularly a human patient.

By the phrase “prevention or reduction of vascular access dysfunction in association with the insertion or repair of an indwelling shunt, fistula or catheter” as used herein, is meant that the incidence of vascular thrombosis and/or fistula failure and/or shunt failure and/or vascular access clotting and/or stenosis and/or restenosis and/or the need for declotting an indwelling vascular access shunt, fistula or catheter in patients treated with a non-peptide TPO receptor agonist collected over the observation period are prevented or reduced in comparison to untreated patients.

By the term “collected over the observation period” as used herein, means a period of up to or about 12 months, preferably 12 months.

An example of damaged tissue, such as flesh wounds, as used herein is restenosis associated with arterial coronary intervention, suitably the insertion of a stent. The current invention is directed to the inhibition of restenosis associated with arterial coronary intervention.

An example of damaged tissue, such as flesh wounds, as used herein is peripheral vascular disease in mammals, including humans. By the term “peripheral vascular disease” and derivatives thereof, as used herein, is meant a non-coronary artery that has undergone percutaneous intervention, with or without stent placement, suitably, the intervention was due to a disease state selected form: renal artery stenosis; in cerebral vessels—carotid artery stenosis and vertebral arteries; and peripheral atherosclerosis in vessels, preferably the internal iliac artery, the femoral artery or in mesenteric vessels. Treatment of peripheral vascular disease with a non-peptide TPO receptor agonist will be similar to the treatment of vascular access dysfunction as described above.

When describing the treatment of damaged tissue, such as flesh wounds, with a non-peptide TPO receptor agonist, as described herein, a favorable result is a decrease in scaring.

When describing treatment, particularly of age damaged tissue, with a non-peptide TPO receptor agonist, as described herein, a favorable result is prolonging the life of the subject.

When describing treatment, particularly of Alzheimer's disease, with a non-peptide TPO receptor agonist, as described herein, a favorable result is the enhancement of memory and/or cognitive function of the subject.

All publications, including but not limited to patents and patent applications, cited in this specification are herein incorporated by reference as though fully set forth.

Compounds of Formula (I) are included in the pharmaceutical compositions of the invention and used in the methods of the invention. Where a —COOH or —OH group is present, pharmaceutically acceptable esters can be employed, for example methyl, ethyl, pivaloyloxymethyl, and the like for —COOH, and acetate maleate and the like for —OH, and those esters known in the art for modifying solubility or hydrolysis characteristics, for use as sustained release or prodrug formulations.

The compounds of Formulas I and II are disclosed and claimed, along with pharmaceutically acceptable salts, hydrates, solvates and esters thereof, as being useful as an agonist of the TPO receptor, particularly in enhancing platelet production and particularly in the treatment of thrombocytopenia, in International Application No. PCT/US01/16863, having an International filing date of May 24, 2001; International Publication Number WO 01/89457 and an International Publication date of Nov. 29, 2001, the entire disclosure of which is hereby incorporated by reference. Compounds of Formulas I and II and pharmaceutically acceptable salts, hydrates, solvates and esters thereof, are prepared as described in International Application No. PCT/US01/16863. The bis-(monoethanolamine) salt of a compound described in International Application No. PCT/US01/16863, is described in International Application No. PCT/US03/16255, having an International filing date of May 21, 2003; International Publication Number WO 03/098992 and an International Publication date of Dec. 4, 2003.

The treatment of degenerative diseases/injuries, as described herein, is accomplished by the administration of a non-peptide TPO receptor agonist and is not limited to any particular mechanism of action. A mechanism of action for treating degenerative diseases/injuries, as described herein, is by stimulating the survival and/or production of stem cells and/or increasing stem cell function and/or longevity to a therapeutic extent.

By the term “co-administering” and derivatives thereof as used herein is meant either simultaneous administration or any manner of separate sequential administration of a TPO receptor agonist, as described herein, and a further active ingredient or ingredients, known to treat degenerative diseases/injuries. Preferably, if the administration is not simultaneous, the compounds are administered in a close time proximity to each other. Furthermore, it does not matter if the compounds are administered in the same dosage form, e.g. one compound may be administered topically and another compound may be administered orally.

Examples of a further active ingredient or ingredients for use in combination with non-peptide TPO receptor agonists according to the present invention include but are not limited to: chemoprotective or myeloprotective agents such as G-CSF, BB10010 (Clemons et al., Breast Cancer Res. Treatment, 1999, 57, 127), amifostine (Ethyol) (Fetscher et al., Current Opinion in Hemat., 2000, 7, 255-60), SCF, IL-11, MCP-4, IL-1-beta, AcSDKP (Gaudron et al., Stem Cells, 1999, 17, 100-6), TNF-a, TGF-b, MIP-1a (Egger et al., Bone Marrow Transpl., 1998, 22 (Suppl. 2), 34-35), and other molecules identified as having anti-apoptotic, survival or proliferative properties.

Tpo has been demonstrated to act as a mobilizer of stem cells into the peripheral blood (Neumann T. A. et al., Cytokines, Cell. & Mol. Ther., 2000, 6, 47-56). This activity can synergize with stem cell mobilizers such as G-CSF (Somolo et al., Blood, 1999, 93, 2798-2806). The TPO receptor agonists of the present invention are useful in increasing the numbers of stem cells in circulation in donors prior to leukapheresis for hematopoietic stem-cell transplantation in patients receiving myelo-ablative chemotherapy.

Likewise, TPO stimulates growth of myeloid cells, particularly those of granulocyte/macrophage lineage (Holly et al., U.S. Pat. No. 5,989,537). Granulocyte/macrophage progenitors are cells of the myeloid lineage that mature as neutrophils, monocytes, basophils and eosinophils. The compounds described in the present invention have therapeutic utility in stimulating the poliferation of neutrophils in patients with neutropenic conditions.

Further, compounds that treat diseases caused by excessive bone loss or cartilage or matrix degradation are known to be used in combination with further active ingredients. (PCT/US03/06147, having an International filing date of Feb. 28, 2003). According to the present invention, non-peptide TPO receptor agonists are useful when administered with further active compounds known to treat diseases caused by excessive bone loss or cartilage or matrix degradation, such as: an organic bisphosphonate, an estrogen receptor modulator, an androgen receptor modulator, an inhibitor of osteoclast proton ATPase, an inhibitor of HMG-CoA reductase, an integrin receptor antagonist, or an osteobalst anabolic agent.

It is part of this discovery that the in vivo administration of non-peptide TPO receptor agonists is useful in treating Parkinson's disease, Huntingtion's disease, multiple sclerosis and ischaemic brain injury. Stem cells, including adult bone marrow stem cells are indicated as effective in treating multiple sclerosis; Stangel M. et al., Progress in Neurobiology, 68(5): 361-76, 2002 Dec. Neural stem cells and their use in Parkinson's disease, Huntingtion's disease, multiple sclerosis and ischaemic brain injury is described in Ostenfield T. et al., Advances & Technical standards in Neurosurgery, 28: 3-89, 2003.

Further, it is part of this discovery that the in vivo administration of non-peptide TPO receptor agonists are useful in the regeneration and repair of tissues that respond to stem cell treatment. Such tissues are readily known or readily ascertainable by those skilled in the art. For example, stem cells are indicated as being useful in treating patients with myocardial infarction, cardiovascular disorders and cardiovascular disease; Stamm C. et al., Lancet. 361(9351): 45-6, 2003 and Semsarian C., Internal Medicine Journal. 32(5-6): 259-65, 2002. Stem cells are indicated in treating, repairing and/or in the regeneration of liver disease/tissue, gastrointestinal disease/tissue and kidney disease/tissue; Choi D. et al., Cell transplantation, 11(4): 359-68, 2002, Poulsom R. et al., Journal of Pathology, 197 (4): 441-56, 2002 and Alison M. et al., Journal of Pathology, 197 (4): 419-23, 2002.

Further, it is part of this discovery that the in vivo administration of non-peptide TPO receptor agonists are useful in the treatment of diabetes/diabetes mellitus. Stem cells are indicated in treating diabetes, Berna G, et al., Biomedicine & Pharmacotherapy, 55(4): 206-12, 2001 and Beilhack G F., et al., Diabetes, 52(1):59-68, 2003.

Additional examples of a further active ingredient or ingredients for use in combination with non-peptide TPO receptor agonists according to the present invention include but are not limited to: stem cell, megakaryocyte, neutrophil mobilizers such as chemotherapeutic agents (i.e., cytoxan, etoposide, cisplatin, Ballestrero A. et al., Oncology, 2000, 59, 7-13), chemokines, IL-8, Gro-beta (King, A. G. et al. J. Immun., 2000, 164, 3774-82), receptor agonist or antagonist antibodies, small molecule cytokine or receptor agonists or antagonists, SCF, Flt3 ligand, adhesion molecule inhibitors or antibodies such as: anti-VLA-4 (Kikuta T. et al., Exp. Hemat., 2000, 28, 311-7) or anti-CD44 (Vermeulen M. et al., Blood, 1998, 92, 894-900), cytokine/chemokine/interleukin or receptor agonist or antagonist antibodies, MCP-4 (Berkhout T A., et al., J. Biol. Chem., 1997, 272, 16404-16413; Uguccioni M. et al., J. Exp. Med., 1996, 183, 2379-2384).

Because the pharmaceutically active compounds of the present invention are active as TPO receptor agonists they exhibit therapeutic utility in treating degenerative diseases/injuries.

Degenerative diseases are known to have many causative factors, including but not limited to, viral infections (including, but not limited to; HIV, hepatitis C, parvovirus) and liver disease, aging, auto immune diseases, neural disease/damage, liver disease/damage, kidney disease/damage, gastrointestinal disease/damage, cardiovascular disease/damage and pancreatic disease/damage. This invention relates to the treatment of degenerative diseases regardless of the factor or factors causing the condition. The pharmaceutically active compounds of this invention are also useful in treating degenerative diseases when the causative factor or factors of the condition are unknown or have yet to be identified.

A skilled physician will be able to determine the appropriate situation in which subjects are susceptible to or at risk of, for example, stroke as well as suffering from stroke for administration by methods of the present invention.

Prophylactic use of the compounds of this invention is contemplated whenever a degenerative disease/injury is anticipated. Prophylactic uses of the compounds of this invention includes but is not limited to transplant surgery, surgery, anesthesia prior to child birth and gut protection.

TPO is known to have various effects including anti-apototic/survival effects on megakaryocytes, platelets and stem cells, and proliferative effects on stem cells and megakaryocytic cells (Kuter D. J. Seminars in Hematology, 2000, 37, 41-9). These TPO activities effectively increase the number of stem and progenitor cells so that there is synergistic effects when TPO is used in conjunction with other cytokines that induce differentiation.

The non-peptide TPO receptor agonists of the current invention are also useful in acting on cells for survival and/or proliferation in conjunction with other agents known to act on cells for survival and/or proliferation. Such other agents include but are not limited to: G-CSF, GM-CSF, TPO, M-CSF, EPO, Gro-beta, IL-11, SCF, FLT3 ligand, LIF, IL-3, IL-6, IL-1, Progenipoietin, NESP, SD-01, or IL-5 or a biologically active derivative of any of the aforementioned agents, KT6352 (Shiotsu Y. et al., Exp. Hemat. 1998, 26, 1195-1201), uteroferrin (Laurenz J C., et al. Comp. Biochem. & Phys., Part A. Physiology., 1997, 116, 369-77), FK23 (Hasegawa T., et al. Int. J. Immunopharm., 1996, 18 103-112) and other molecules identified as having anti-apoptotic, survival or proliferative properties for stem cells, progenitor cells, or other cells expressing TPO Receptors.

The non-peptide TPO receptor agonist of this invention interact differently at the TPO receptor than does TPO. One result of this differing interaction is that the non-peptide TPO receptor agonist of this invention are useful in combination with TPO.

One skilled in the art can readily determine by known methods if a compound is a non-peptide TPO receptor agonist and thus included within the scope of the current invention. By way of example, the following assays can be employed:

### Luciferase Assay

Compounds are tested for potency as agonists of the TPO receptor in a Luciferase assay such as described in Lamb, et al., Nucleic Acids Research 23: 3283-3289 (1995) and Seidel, et al., Proc. Natl. Acad. Sci., USA 92: 3041-3045 (1995) by substituting a TPO-responsive BaF3 cell line (Vigon et al. Proc. Natl. Acad. Sci. USA 1992, 89, 5640-5644) for the HepG2 cells utilized therein. The murine BaF3 cells express TPO receptors and closely match the pattern of STAT (signal transducers and activators of transcription) activation observed in primary murine and human bone marrow cells.

### Proliferation Assay

Compounds are tested in an in vitro proliferation assay using the human UT7TPO cell line. UT7TPO cells are a human megakaryoblastic cell line that express Tpo-R, whose survival and growth is dependent on the presence of TPO (Komatsu et al. Blood 1996, 87, 4552).

### Differentiation Assay

Compounds are tested for their ability in stimulating the maturation of megakaryocytes from human bone marrow cells. In this assay, purified human CD34+ progenitor cells are incubated in liquid culture with test compounds for 10 days and the number of cells expressing the transmembrane glycoprotein CD41 (gpllb), a megakaryocytic marker, is then measured by flow cytometry (see Cwirla, S. E. et al Science, 1997, 276, 1696).

The pharmaceutically active compounds within the scope of this invention are useful as non-peptide TPO receptor agonists in mammals, particularly humans, in need thereof.

The ability of non-peptide TPO receptor agonists to treat degenerative diseases/injuries is demonstrated by activity in the CD34+ Progenitor Cell Proliferation Assay.

### CD34+ Progenitor Cell Proliferation Assay

Compounds are tested for their ability in stimulating the survival and proliferation of early CD34+ progenitor cells from human bone marrow. In this assay, purified human CD34+ progenitor cells are incubated in liquid culture with test compounds for up to 7 days and the number of cells expressing the early stem cell marker CD34 are then measured by flow cytometry and compared to untreated cells (see Liu et al. Bone Marrow Transplantation. 24:247-52, 1999). The compound 3′-{N′-[3-methyl-5-oxo-1-(4-trifluoromethylphenyl)-1,5-dihydropyrazol-4-ylidene]hydrazino}-2′-hydroxybiphenyl-3-carboxylic acid was tested in the CD34+ Progenitor Cell Proliferation Assay and at 3 uM increased the number of CD34+ cells in liquid culture by up to 2-fold over vehicle controls at days 2, 5 and 7. rhTpo (100 ng/ml) also demonstrated a 2-fold increase in the number of CD34+ cells in this experiment.

### CD34+ Progenitor Cell Proliferation Assay Experiment 2

### Method:

Human marrow progenitor CD34+ cells were washed in SFEM, counted and brought to 2×105/mL in 24-well plates and incubated for 7 days in the presence of compound or rhTpo. Dilutions of 3′-{N′-[1-(3,4-Dimethylphenyl)-3-methyl-5-oxo-1,5-dihydropyrazol-4-ylidene]hydrazino}-2′-hydroxybiphenyl-3-carboxylic acid bis-(monoethanolamine) were made in distilled water to equal 0, 0.3, 1 and 3 uM final concentration. Dilutions of 3-{N′-[1-(3,4-dimethylphenyl)-3-methyl-5-oxo-1,5-dihydropyrazol-4-ylidene]hydrazino}-2-hydroxy-3′-tetrazol-5-ylbiphenyl disodium salt were made in DMSO to equal 0, 0.3, 1 and 3 uM final concentration in 0.1% DMSO. Recombinant human TPO at a final concentration of 100 ng/mL was used as a positive control. At day 7, cells were counted and flow cytometry was performed using FITC-CD34.

### Results:

3′-{N′-[1-(3,4-Dimethylphenyl)-3-methyl-5-oxo-1,5-dihydropyrazol-4-ylidene]hydrazino}-2′-hydroxybiphenyl-3-carboxylic acid bis-(monoethanolamine) at 3 uM increased the number of CD34+ cells by 3-fold.

rhTpo (100 ng/ml) also demonstrated a 15-fold increase in the number of CD34+ cells in this experiment.

{N′-[1-(3,4-dimethylphenyl)-3-methyl-5-oxo-1,5-dihydropyrazol-4-ylidene]hydrazino}-2-hydroxy-3′-tetrazol-5-ylbiphenyl disodium salt at 3 uM increased the number of CD34+ cells by 3.6-fold.

rhTpo (100 ng/ml) also demonstrated a 3.4-fold increase in the number of CD34+ cells in this experiment.

Increased activity of CD34+ released into blood from marrow by mobilization has been indicated in the treatment of cardiovascular disease. (Experimental Hematology 2008; 36:687-694)

Some of the compounds within the scope of the invention were tested and showed activation from about 4% to 100% of control at a concentration of 0.001-10 uM in the luciferase assay. Some of the compounds of the invention also promoted the proliferation of 32D-mpl cells at a concentration of 0.003 to 30 uM. Some of the compounds of the invention also showed activity in the CD41 megakaryocytic assay at a concentration of 0.003 to 30 uM.

The present invention therefore provides a method of treating a disease/injury state selected from: nervous system disorders, including transverse myelitis, multiple sclerosis, demyelination occurring after trauma to the brain or spinal cord, acute brain injury, head trauma, spinal cord injury, peripheral nerve injury, ischaemic brain injury, hereditary myelin disorder of the CNS, epilepsy, perinatal asphxia, asphyxia, anoxia, status epilepticus, and stroke; baldness, such as male pattern baldness and alopecia areata; neurodegenerative diseases, such as Alzheimer's disease, Parkinson disease, Huntington's disease, and amyotrophic lateral sclerosis; in the treatment, repair and/or regeneration of tissue, for example: in cardiovascular disorders, myocardial infarction and cardiovascular disease/tissue (hereinafter cardiovascular disease), and in the treatment, repair and/or regeneration of liver disease/tissue (hereinafter liver disease), gastrointestinal disease/tissue (hereinafter gastrointestinal disease) and kidney disease/tissue (hereinafter kidney disease); in the restoration of damaged tissue, such as healing flesh wounds, regenerating age damaged cells and regenerating age damaged tissue; in the treatment of lupus; and in the treatment of diabetes/diabetes mellitus which comprises the administration an effective amount of a non-peptide TPO receptor agonist.

The present invention also provides a method of treating degenerative diseases caused by excessive bone loss or cartilage or matrix degradation, such as: osteoporosis, glucocorticoid induced osteoporosis, Paget's disease, abnormally increased bone turnover, periodontal disease, gingivitis, tooth loss, bone fractures, arthritis, rheumatoid arthritis, osteoarthritis, periprosthetic osteolysis, osteogenesis imperfecta, or metastatic bone disease, which comprises the administration of an effective amount of a non-peptide TPO receptor agonist.

The present invention also provides a method of treating degenerative diseases of the eye such as: macular degeneration, dry eye syndrome, cataracts, diabetic retinopathy, glaucoma, vitreous disease and retinal degeneration, which comprises the administration of an effective amount of a non-peptide TPO receptor agonist.

The present invention also provides a method of treating AIDS, which comprises the administration of an effective amount of a non-peptide TPO receptor agonist.

Further, it is known that certain pharmaceutically active compounds can cause injury to cardiovascular tissue as an undesirable side effect. (Circulation. 2006; 113:2211-2220), (Nat. Rev, Cancer, Vol. 7, May 2007: 332-344), (Expert Rev. Cardiovasc. Ther. 6(7), 2008). The TPO receptor agonists of the invention are useful in treating and/or reparing the cardiovascular disease that results from the administration of such pharmaceutically active compounds.

Suitably, the pharmaceutically active compound is an anti-neoplastic compound.

Anti-neoplastic agents that can cause injury to cardiovascular tissue for which a TPO receptor agonist of the invention would be useful in treating the resultant cardiovascular disease include, but are not limited to, anti-microtubule agents such as diterpenoids and vinca alkaloids; platinum coordination complexes; alkylating agents such as nitrogen mustards, oxazaphosphorines, alkylsulfonates, nitrosoureas, and triazenes; antibiotic agents such as anthracyclins, actinomycins and bleomycins; topoisomerase II inhibitors such as epipodophyllotoxins; antimetabolites such as purine and pyrimidine analogues and anti-folate compounds; topoisomerase I inhibitors such as camptothecins; hormones and hormonal analogues; signal transduction pathway inhibitors; non-receptor tyrosine kinase angiogenesis inhibitors; immunotherapeutic agents; proapoptotic agents; cell cycle signaling inhibitors; and chemotherapeutic agents.

More specifically, the following anti-neoplastic agents can cause injury to cardiovascular tissue for which a TPO receptor agonist of the invention would be useful in treating the resultant cardiovascular disease.

Anti-microtubule or anti-mitotic agents are phase specific agents active against the microtubules of tumor cells during M or the mitosis phase of the cell cycle. Examples of anti-microtubule agents include, but are not limited to, diterpenoids and vinca alkaloids.

Diterpenoids, which are derived from natural sources, are phase specific anti-cancer agents that operate at the G₂/M phases of the cell cycle. It is believed that the diterpenoids stabilize the β-tubulin subunit of the microtubules, by binding with this protein. Disassembly of the protein appears then to be inhibited with mitosis being arrested and cell death following. Examples of diterpenoids include, but are not limited to, paclitaxel and its analog docetaxel.

Paclitaxel, 5,20-epoxy-1,2α,4,7β,10β,13α-hexa-hydroxytax-11-en-9-one 4,10-diacetate 2-benzoate 13-ester with (2R,3S)—N-benzoyl-3-phenylisoserine; is a natural diterpene product isolated from the Pacific yew tree Taxus brevifolia and is commercially available as an injectable solution TAXOL®. It is a member of the taxane family of terpenes. It was first isolated in 1971 by Wani et al. J. Am. Chem, Soc., 93:2325. 1971), who characterized its structure by chemical and X-ray crystallographic methods. One mechanism for its activity relates to paclitaxel's capacity to bind tubulin, thereby inhibiting cancer cell growth. Schiff et al., Proc. Natl, Acad, Sci. USA, 77:1561-1565 (1980); Schiff et al., Nature, 277:665-667 (1979); Kumar, J. Biol, Chem, 256: 10435-10441 (1981). For a review of synthesis and anticancer activity of some paclitaxel derivatives see: D. G. I. Kingston et al., Studies in Organic Chemistry vol. 26, entitled “New trends in Natural Products Chemistry 1986”, Attaur-Rahman, P. W. Le Quesne, Eds. (Elsevier, Amsterdam, 1986) pp 219-235.

Paclitaxel has been approved for clinical use in the treatment of refractory ovarian cancer in the United States (Markman et al., Yale Journal of Biology and Medicine, 64:583, 1991; McGuire et al., Ann. Intern, Med., 111:273, 1989) and for the treatment of breast cancer (Holmes et al., J. Nat. Cancer Inst., 83:1797, 1991.) It is a potential candidate for treatment of neoplasms in the skin (Einzig et. al., Proc. Am. Soc. Clin. Oncol., 20:46) and head and neck carcinomas (Forastire et. al., Sem. Oncol., 20:56, 1990). The compound also shows potential for the treatment of polycystic kidney disease (Woo et. al., Nature, 368:750. 1994), lung cancer and malaria. Treatment of patients with paclitaxel results in bone marrow suppression (multiple cell lineages, Ignoff, R. J. et. al, Cancer Chemotherapy Pocket Guide, 1998) related to the duration of dosing above a threshold concentration (50 nM) (Kearns, C. M. et. al., Seminars in Oncology, 3(6) p. 16-23, 1995).

Docetaxel, (2R,3S)—N-carboxy-3-phenylisoserine,N-tert-butyl ester, 13-ester with 5β-20-epoxy-1,2α,4,7β,10β,13α-hexahydroxytax-11-en-9-one 4-acetate 2-benzoate, trihydrate; is commercially available as an injectable solution as TAXOTERE®. Docetaxel is indicated for the treatment of breast cancer. Docetaxel is a semisynthetic derivative of paclitaxel q.v., prepared using a natural precursor, 10-deacetyl-baccatin III, extracted from the needle of the European Yew tree. The dose limiting toxicity of docetaxel is neutropenia.

Vinca alkaloids are phase specific anti-neoplastic agents derived from the periwinkle plant. Vinca alkaloids act at the M phase (mitosis) of the cell cycle by binding specifically to tubulin. Consequently, the bound tubulin molecule is unable to polymerize into microtubules. Mitosis is believed to be arrested in metaphase with cell death following. Examples of vinca alkaloids include, but are not limited to, vinblastine, vincristine, and vinorelbine.

Vinblastine, vincaleukoblastine sulfate, is commercially available as VELBAN® as an injectable solution. Although, it has possible indication as a second line therapy of various solid tumors, it is primarily indicated in the treatment of testicular cancer and various lymphomas including Hodgkin's Disease; and lymphocytic and histiocytic lymphomas. Myelosuppression is the dose limiting side effect of vinblastine.

Vincristine, vincaleukoblastine, 22-oxo-, sulfate, is commercially available as ONCOVIN® as an injectable solution. Vincristine is indicated for the treatment of acute leukemias and has also found use in treatment regimens for Hodgkin's and non-Hodgkin's malignant lymphomas. Alopecia and neurologic effects are the most common side effect of vincristine and to a lesser extent myelosupression and gastrointestinal mucositis effects occur.

Vinorelbine, 3′,4′-didehydro-4′-deoxy-C′-norvincaleukoblastine [R—(R*,R*)-2,3-dihydroxybutanedioate (1:2)(salt)], commercially available as an injectable solution of vinorelbine tartrate (NAVELBINE®), is a semisynthetic vinca alkaloid. Vinorelbine is indicated as a single agent or in combination with other chemotherapeutic agents, such as cisplatin, in the treatment of various solid tumors, particularly non-small cell lung, advanced breast, and hormone refractory prostate cancers. Myelosuppression is the most common dose limiting side effect of vinorelbine.

Platinum coordination complexes are non-phase specific anti-cancer agents, which are interactive with DNA. The platinum complexes enter tumor cells, undergo, aquation and form intra- and interstrand crosslinks with DNA causing adverse biological effects to the tumor. Examples of platinum coordination complexes include, but are not limited to, cisplatin and carboplatin.

Cisplatin, cis-diamminedichloroplatinum, is commercially available as PLATINOL® as an injectable solution. Cisplatin is primarily indicated in the treatment of metastatic testicular and ovarian cancer and advanced bladder cancer. The primary dose limiting side effects of cisplatin are nephrotoxicity, which may be controlled by hydration and diuresis, and ototoxicity.

Carboplatin, platinum, diammine [1,1-cyclobutane-dicarboxylate(2-)—O,O′], is commercially available as PARAPLATIN® as an injectable solution. Carboplatin is primarily indicated in the first and second line treatment of advanced ovarian carcinoma. Bone marrow suppression is the dose limiting toxicity of carboplatin.

Alkylating agents are non-phase anti-cancer specific agents and strong electrophiles. Typically, alkylating agents form covalent linkages, by alkylation, to DNA through nucleophilic moieties of the DNA molecule such as phosphate, amino, sulfhydryl, hydroxyl, carboxyl, and imidazole groups. Such alkylation disrupts nucleic acid function leading to cell death. Examples of alkylating agents include, but are not limited to, nitrogen mustards such as cyclophosphamide, melphalan, and chlorambucil; alkyl sulfonates such as busulfan; nitrosoureas such as carmustine; and triazenes such as dacarbazine.

Cyclophosphamide, 2-[bis(2-chloroethyl)amino]tetrahydro-2H-1,3,2-oxazaphosphorine 2-oxide monohydrate, is commercially available as an injectable solution or tablets as CYTOXAN®. Cyclophosphamide is indicated as a single agent or in combination with other chemotherapeutic agents, in the treatment of malignant lymphomas, multiple myeloma, and leukemias. Alopecia, nausea, vomiting and leukopenia are the most common dose limiting side effects of cyclophosphamide.

Melphalan, 4-[bis(2-chloroethyl)amino]-L-phenylalanine, is commercially available as an injectable solution or tablets as ALKERAN®. Melphalan is indicated for the palliative treatment of multiple myeloma and non-resectable epithelial carcinoma of the ovary. Bone marrow suppression is the most common dose limiting side effect of melphalan.

Chlorambucil, 4-[bis(2-chloroethyl)amino]benzenebutanoic acid, is commercially available as LEUKERAN® tablets. Chlorambucil is indicated for the palliative treatment of chronic lymphatic leukemia, and malignant lymphomas such as lymphosarcoma, giant follicular lymphoma, and Hodgkin's disease. Bone marrow suppression is the most common dose limiting side effect of chlorambucil.

Busulfan, 1,4-butanediol dimethanesulfonate, is commercially available as MYLERAN® TABLETS. Busulfan is indicated for the palliative treatment of chronic myelogenous leukemia. Bone marrow suppression is the most common dose limiting side effects of busulfan.

Carmustine, 1,3-[bis(2-chloroethyl)-1-nitrosourea, is commercially available as single vials of lyophilized material as BiCNU®. Carmustine is indicated for the palliative treatment as a single agent or in combination with other agents for brain tumors, multiple myeloma, Hodgkin's disease, and non-Hodgkin's lymphomas. Delayed myelosuppression is the most common dose limiting side effects of carmustine.

Dacarbazine, 5-(3,3-dimethyl-1-triazeno)-imidazole-4-carboxamide, is commercially available as single vials of material as DTIC-Dome®. Dacarbazine is indicated for the treatment of metastatic malignant melanoma and in combination with other agents for the second line treatment of Hodgkin's Disease. Nausea, vomiting, and anorexia are the most common dose limiting side effects of dacarbazine.

Antibiotic anti-neoplastics are non-phase specific agents, which bind or intercalate with DNA. Typically, such action results in stable DNA complexes or strand breakage, which disrupts ordinary function of the nucleic acids leading to cell death. Examples of antibiotic anti-neoplastic agents include, but are not limited to, actinomycins such as dactinomycin, anthrocyclins such as daunorubicin and doxorubicin; and bleomycins.

Dactinomycin, also know as Actinomycin D, is commercially available in injectable form as COSMEGEN®. Dactinomycin is indicated for the treatment of Wilm's tumor and rhabdomyosarcoma. Nausea, vomiting, and anorexia are the most common dose limiting side effects of dactinomycin.

Daunorubicin, (8S-cis-)-8-acetyl-10-[(3-amino-2,3,6-trideoxy-α-L-Iyxo-hexopyranosyl)oxy]-7,8,9,10-tetrahydro-6,8,11-trihydroxy-1-methoxy-5,12 naphthacenedione hydrochloride, is commercially available as a liposomal injectable form as DAUNOXOME® or as an injectable as CERUBIDINE®. Daunorubicin is indicated for remission induction in the treatment of acute nonlymphocytic leukemia and advanced HIV associated Kaposi's sarcoma. Myelosuppression is the most common dose limiting side effect of daunorubicin.

Doxorubicin, (8S,10S)-10-[(3-amino-2,3,6-trideoxy-α-L-Iyxo-hexopyranosyl)oxy]-8-glycoloyl, 7,8,9,10-tetrahydro-6,8,11-trihydroxy-1-methoxy-5,12 naphthacenedione hydrochloride, is commercially available as an injectable form as RUBEX® or ADRIAMYCIN RDF®. Doxorubicin is primarily indicated for the treatment of acute lymphoblastic leukemia and acute myeloblastic leukemia, but is also a useful component in the treatment of some solid tumors and lymphomas. Myelosuppression is the most common dose limiting side effect of doxorubicin.

Bleomycin, a mixture of cytotoxic glycopeptide antibiotics isolated from a strain of Streptomyces verticillus, is commercially available as BLENOXANE®. Bleomycin is indicated as a palliative treatment, as a single agent or in combination with other agents, of squamous cell carcinoma, lymphomas, and testicular carcinomas. Pulmonary and cutaneous toxicities are the most common dose limiting side effects of bleomycin.

Topoisomerase II inhibitors include, but are not limited to, epipodophyllotoxins.

Epipodophyllotoxins are phase specific anti-neoplastic agents derived from the mandrake plant. Epipodophyllotoxins typically affect cells in the S and G₂ phases of the cell cycle by forming a ternary complex with topoisomerase II and DNA causing DNA strand breaks. The strand breaks accumulate and cell death follows. Examples of epipodophyllotoxins include, but are not limited to, etoposide and teniposide.

Etoposide, 4′-demethyl-epipodophyllotoxin 9[4,6-0-(R)-ethylidene-β-D-glucopyranoside], is commercially available as an injectable solution or capsules as VePESID® and is commonly known as VP-16. Etoposide is indicated as a single agent or in combination with other chemotherapy agents in the treatment of testicular and non-small cell lung cancers. Myelosuppression is the most common side effect of etoposide. The incidence of leucopenia tends to be more severe than thrombocytopenia.

Teniposide, 4′-demethyl-epipodophyllotoxin 9[4,6-0-(R)-thenylidene-β-D-glucopyranoside], is commercially available as an injectable solution as VUMON® and is commonly known as VM-26. Teniposide is indicated as a single agent or in combination with other chemotherapy agents in the treatment of acute leukemia in children. Myelosuppression is the most common dose limiting side effect of teniposide. Teniposide can induce both leucopenia and thrombocytopenia.

Antimetabolite neoplastic agents are phase specific anti-neoplastic agents that act at S phase (DNA synthesis) of the cell cycle by inhibiting DNA synthesis or by inhibiting purine or pyrimidine base synthesis and thereby limiting DNA synthesis. Consequently, S phase does not proceed and cell death follows. Examples of antimetabolite anti-neoplastic agents include, but are not limited to, fluorouracil, methotrexate, cytarabine, mercaptopurine, thioguanine, and gemcitabine.

5-fluorouracil, 5-fluoro-2,4-(1H,3H) pyrimidinedione, is commercially available as fluorouracil. Administration of 5-fluorouracil leads to inhibition of thymidylate synthesis and is also incorporated into both RNA and DNA. The result typically is cell death. 5-fluorouracil is indicated as a single agent or in combination with other chemotherapy agents in the treatment of carcinomas of the breast, colon, rectum, stomach and pancreas. Myelosuppression and mucositis are dose limiting side effects of 5-fluorouracil. Other fluoropyrimidine analogs include 5-fluoro deoxyuridine (floxuridine) and 5-fluorodeoxyuridine monophosphate.

Cytarabine, 4-amino-1-β-D-arabinofuranosyl-2 (1H)-pyrimidinone, is commercially available as CYTOSAR-U® and is commonly known as Ara-C. It is believed that cytarabine exhibits cell phase specificity at S-phase by inhibiting DNA chain elongation by terminal incorporation of cytarabine into the growing DNA chain. Cytarabine is indicated as a single agent or in combination with other chemotherapy agents in the treatment of acute leukemia. Other cytidine analogs include 5-azacytidine and 2′,2′-difluorodeoxycytidine (gemcitabine). Cytarabine induces leucopenia, thrombocytopenia, and mucositis.

Mercaptopurine, 1,7-dihydro-6H-purine-6-thione monohydrate, is commercially available as PURINETHOL®. Mercaptopurine exhibits cell phase specificity at S-phase by inhibiting DNA synthesis by an as of yet unspecified mechanism. Mercaptopurine is indicated as a single agent or in combination with other chemotherapy agents in the treatment of acute leukemia. Myelosuppression and gastrointestinal mucositis are expected side effects of mercaptopurine at high doses. A useful mercaptopurine analog is azathioprine.

Thioguanine, 2-amino-1,7-dihydro-6H-purine-6-thione, is commercially available as TABLOID®. Thioguanine exhibits cell phase specificity at S-phase by inhibiting DNA synthesis by an as of yet unspecified mechanism. Thioguanine is indicated as a single agent or in combination with other chemotherapy agents in the treatment of acute leukemia. Myelosuppression, including leucopenia, thrombocytopenia, and anemia, is the most common dose limiting side effect of thioguanine administration. However, gastrointestinal side effects occur and can be dose limiting. Other purine analogs include pentostatin, erythrohydroxynonyladenine, fludarabine phosphate, and cladribine.

Gemcitabine, 2′-deoxy-2′,2′-difluorocytidine monohydrochloride (β-isomer), is commercially available as GEMZAR®. Gemcitabine exhibits cell phase specificity at S-phase and by blocking progression of cells through the G1/S boundary. Gemcitabine is indicated in combination with cisplatin in the treatment of locally advanced non-small cell lung cancer and alone in the treatment of locally advanced pancreatic cancer. Myelosuppression, including leucopenia, thrombocytopenia, and anemia, is the most common dose limiting side effect of gemcitabine administration.

Methotrexate, N-[4[[(2,4-diamino-6-pteridinyl)methyl]methylamino]benzoyl]-L-glutamic acid, is commercially available as methotrexate sodium. Methotrexate exhibits cell phase effects specifically at S-phase by inhibiting DNA synthesis, repair and/or replication through the inhibition of dyhydrofolic acid reductase which is required for synthesis of purine nucleotides and thymidylate. Methotrexate is indicated as a single agent or in combination with other chemotherapy agents in the treatment of choriocarcinoma, meningeal leukemia, non-Hodgkin's lymphoma, and carcinomas of the breast, head, neck, ovary and bladder. Myelosuppression (leucopenia, thrombocytopenia, and anemia) and mucositis are expected side effect of methotrexate administration.

Camptothecins, including, camptothecin and camptothecin derivatives are available or under development as Topoisomerase I inhibitors. Camptothecins cytotoxic activity is believed to be related to its Topoisomerase I inhibitory activity. Examples of camptothecins include, but are not limited to irinotecan, topotecan, and the various optical forms of 7-(4-methylpiperazino-methylene)-10,11-ethylenedioxy-20-camptothecin described below.

Irinotecan HCl, (4S)-4,11-diethyl-4-hydroxy-9-[(4-piperidinopiperidino) carbonyloxy]-1H-pyrano[3′,4′,6,7]indolizino[1,2-b]quinoline-3,14(4H,12H)-dione hydrochloride, is commercially available as the injectable solution CAMPTOSAR®.

Irinotecan is a derivative of camptothecin which binds, along with its active metabolite SN-38, to the topoisomerase I-DNA complex. It is believed that cytotoxicity occurs as a result of irreparable double strand breaks caused by interaction of the topoisomerase I: DNA: irintecan or SN-38 ternary complex with replication enzymes. Irinotecan is indicated for treatment of metastatic cancer of the colon or rectum. The dose limiting side effects of irinotecan HCl are myelosuppression, including neutropenia, and GI effects, including diarrhea.

Topotecan HCl, (S)-10-[(dimethylamino)methyl]-4-ethyl-4,9-dihydroxy-1H-pyrano[3′,4′,6,7]indolizino[1,2-b]quinoline-3,14-(4H,12H)-dione monohydrochloride, is commercially available as the injectable solution HYCAMTIN®. Topotecan is a derivative of camptothecin which binds to the topoisomerase I-DNA complex and prevents religation of singles strand breaks caused by Topoisomerase I in response to torsional strain of the DNA molecule. Topotecan is indicated for second line treatment of metastatic carcinoma of the ovary and small cell lung cancer. The dose limiting side effect of topotecan HCl is myelosuppression, primarily neutropenia.

The camptothecin derivative of formula A following, currently under development, including the racemic mixture (R,S) form as well as the R and S enantiomers:

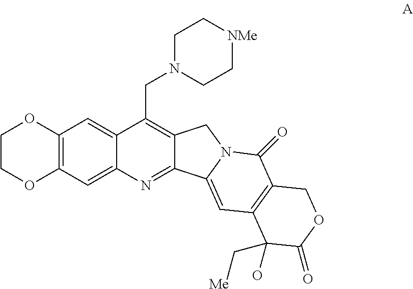

known by the chemical name “7-(4-methylpiperazino-methylene)-10,11-ethylenedioxy-20(R,S)-camptothecin (racemic mixture) or “7-(4-methylpiperazino-methylene)-10,11-ethylenedioxy-20(R)-camptothecin (R enantiomer) or “7-(4-methylpiperazino-methylene)-10,11-ethylenedioxy-20(S)-camptothecin (S enantiomer). Such compound as well as related compounds are described, including methods of making, in U.S. Pat. Nos. 6,063,923; 5,342,947; 5,559,235; 5,491,237 and pending U.S. patent application Ser. No. 08/977,217 filed Nov. 24, 1997.

Hormones and hormonal analogues are useful compounds for treating cancers in which there is a relationship between the hormone(s) and growth and/or lack of growth of the cancer. Examples of hormones and hormonal analogues useful in cancer treatment include, but are not limited to, adrenocorticosteroids such as prednisone and prednisolone which are useful in the treatment of malignant lymphoma and acute leukemia in children; aminoglutethimide and other aromatase inhibitors such as anastrozole, letrazole, vorazole, and exemestane useful in the treatment of adrenocortical carcinoma and hormone dependent breast carcinoma containing estrogen receptors; progestrins such as megestrol acetate useful in the treatment of hormone dependent breast cancer and endometrial carcinoma; estrogens, androgens, and anti-androgens such as flutamide, nilutamide, bicalutamide, cyproterone acetate and 5α-reductases such as finasteride and dutasteride, useful in the treatment of prostatic carcinoma and benign prostatic hypertrophy; anti-estrogens such as tamoxifen, toremifene, raloxifene, droloxifene, iodoxyfene, as well as selective estrogen receptor modulators (SERMS) such those described in U.S. Pat. Nos. 5,681,835, 5,877,219, and 6,207,716, useful in the treatment of hormone dependent breast carcinoma and other susceptible cancers; and gonadotropin-releasing hormone (GnRH) and analogues thereof which stimulate the release of leutinizing hormone (LH) and/or follicle stimulating hormone (FSH) for the treatment prostatic carcinoma, for instance, LHRH agonists and antagagonists such as goserelin acetate and luprolide.

Signal transduction pathway inhibitors are those inhibitors, which block or inhibit a chemical process which evokes an intracellular change. As used herein this change is cell proliferation or differentiation. Signal tranduction inhibitors useful in the present invention include inhibitors of receptor tyrosine kinases, non-receptor tyrosine kinases, SH2/SH3 domain blockers, serine/threonine kinases, phosphotidyl inositol-3 kinases, myo-inositol signaling, and Ras oncogenes.

Several protein tyrosine kinases catalyse the phosphorylation of specific tyrosyl residues in various proteins involved in the regulation of cell growth. Such protein tyrosine kinases can be broadly classified as receptor or non-receptor kinases.

Receptor tyrosine kinases are transmembrane proteins having an extracellular ligand binding domain, a transmembrane domain, and a tyrosine kinase domain. Receptor tyrosine kinases are involved in the regulation of cell growth and are generally termed growth factor receptors. Inappropriate or uncontrolled activation of many of these kinases, i.e. aberrant kinase growth factor receptor activity, for example by over-expression or mutation, has been shown to result in uncontrolled cell growth. Accordingly, the aberrant activity of such kinases has been linked to malignant tissue growth. Consequently, inhibitors of such kinases could provide cancer treatment methods. Growth factor receptors include, for example, epidermal growth factor receptor (EGFr), platelet derived growth factor receptor (PDGFr), erbB2, erbB4, vascular endothelial growth factor receptor (VEGFr), tyrosine kinase with immunoglobulin-like and epidermal growth factor homology domains (TIE-2), insulin growth factor-I (IGFI) receptor, macrophage colony stimulating factor (cfms), BTK, ckit, cmet, fibroblast growth factor (FGF) receptors, Trk receptors (TrkA, TrkB, and TrkC), ephrin (eph) receptors, and the RET protooncogene. Several inhibitors of growth receptors are under development and include ligand antagonists, antibodies, tyrosine kinase inhibitors and anti-sense oligonucleotides. Growth factor receptors and agents that inhibit growth factor receptor function are described, for instance, in Kath, John C., Exp. Opin. Ther. Patents (2000) 10(6):803-818; Shawver et al DDT Vol 2, No. 2 Feb. 1997; and Lofts, F. J. et al, “Growth factor receptors as targets”, New Molecular Targets for Cancer Chemotherapy, ed. Workman, Paul and Kerr, David, CRC press 1994, London.

Tyrosine kinases, which are not growth factor receptor kinases are termed non-receptor tyrosine kinases. Non-receptor tyrosine kinases for use in the present invention, which are targets or potential targets of anti-cancer drugs, include cSrc, Lck, Fyn, Yes, Jak, cAbl, FAK (Focal adhesion kinase), Brutons tyrosine kinase, and Bcr-Abl. Such non-receptor kinases and agents which inhibit non-receptor tyrosine kinase function are described in Sinh, S, and Corey, S. J., (1999) Journal of Hematotherapy and Stem Cell Research 8 (5): 465-80; and Bolen, J. B., Brugge, J. S., (1997) Annual review of Immunology. 15: 371-404.

SH2/SH3 domain blockers are agents that disrupt SH2 or SH3 domain binding in a variety of enzymes or adaptor proteins including, PI3-K p85 subunit, Src family kinases, adaptor molecules (Shc, Crk, Nck, Grb2) and Ras-GAP. SH2/SH3 domains as targets for anti-cancer drugs are discussed in Smithgall, T. E. (1995), Journal of Pharmacological and Toxicological Methods. 34(3) 125-32.

Inhibitors of Serine/Threonine Kinases including MAP kinase cascade blockers which include blockers of Raf kinases (rafk), Mitogen or Extracellular Regulated Kinase (MEKs), and Extracellular Regulated Kinases (ERKs); and Protein kinase C family member blockers including blockers of PKCs (alpha, beta, gamma, epsilon, mu, lambda, iota, zeta). IkB kinase family (IKKa, IKKb), PKB family kinases, akt kinase family members, and TGF beta receptor kinases. Such Serine/Threonine kinases and inhibitors thereof are described in Yamamoto, T., Taya, S., Kaibuchi, K., (1999), Journal of Biochemistry. 126 (5) 799-803; Brodt, P, Samani, A., and Navab, R. (2000), Biochemical Pharmacology, 60. 1101-1107; Massague, J., Weis-Garcia, F. (1996) Cancer Surveys. 27:41-64; Philip, P. A., and Harris, A. L. (1995), Cancer Treatment and Research. 78: 3-27, Lackey, K. et al Bioorganic and Medicinal Chemistry Letters, (10), 2000, 223-226; U.S. Pat. No. 6,268,391; and Martinez-lacaci, L., et al, Int. J. Cancer (2000), 88(1), 44-52.

Inhibitors of Phosphotidyl inositol-3 Kinase family members including blockers of PI3-kinase, ATM, DNA-PK, and Ku. Such kinases are discussed in Abraham, R. T. (1996), Current Opinion in Immunology. 8 (3) 412-8; Canman, C. E., Lim, D. S. (1998), Oncogene 17 (25) 3301-3308; Jackson, S. P. (1997), International Journal of Biochemistry and Cell Biology. 29 (7):935-8; and Zhong, H. et al, Cancer res, (2000) 60(6), 1541-1545.

Myo-inositol signaling inhibitors such as phospholipase C blockers and Myoinositol analogues. Such signal inhibitors are described in Powis, G., and Kozikowski A., (1994) New Molecular Targets for Cancer Chemotherapy ed., Paul Workman and David Kerr, CRC press 1994, London.

Another group of signal transduction pathway inhibitors are inhibitors of Ras Oncogene. Such inhibitors include inhibitors of farnesyltransferase, geranyl-geranyl transferase, and CAAX proteases as well as anti-sense oligonucleotides, ribozymes and immunotherapy. Such inhibitors have been shown to block ras activation in cells containing wild type mutant ras, thereby acting as antiproliferation agents. Ras oncogene inhibition is discussed in Scharovsky, O. G., Rozados, V. R., Gervasoni, S. I. Matar, P. (2000), Journal of Biomedical Science. 7(4) 292-8; Ashby, M. N. (1998), Current Opinion in Lipidology. 9 (2) 99-102; and BioChim. Biophys. Acta, (19899) 1423(3):19-30.

As mentioned above, antibody antagonists to receptor kinase ligand binding may also serve as signal transduction inhibitors. This group of signal transduction pathway inhibitors includes the use of humanized antibodies to the extracellular ligand binding domain of receptor tyrosine kinases. For example lmclone C225 EGFR specific antibody (see Green, M. C. et al, Monoclonal Antibody Therapy for Solid Tumors, Cancer Treat. Rev., (2000), 26(4), 269-286); Herceptin® erbB2 antibody (see Tyrosine Kinase Signalling in Breast cancer:erbB Family Receptor Tyrosine Kniases, Breast cancer Res., 2000, 2(3), 176-183); and 2CB VEGFR2 specific antibody (see Brekken, R. A. et al, Selective Inhibition of VEGFR2 Activity by a monoclonal Anti-VEGF antibody blocks tumor growth in mice, Cancer Res. (2000) 60, 5117-5124).

Inhibitors of angiogenesis related VEGFR and TIE2 are discussed above in regard to signal transduction inhibitors (both receptors are receptor tyrosine kinases). Angiogenesis in general is linked to erbB2/EGFR signaling since inhibitors of erbB2 and EGFR have been shown to inhibit angiogenesis, primarily VEGF expression. (Bruns C J et al (2000), Cancer Res., 60: 2926-2935; Schreiber A B, Winkler M E, and Derynck R. (1986), Science, 232: 1250-1253; Yen L et al. (2000), Oncogene 19: 3460-3469).

There are a number of immunologic strategies to generate an immune response. These strategies are generally in the realm of tumor vaccinations. The efficacy of immunologic approaches may be greatly enhanced through combined inhibition of signaling pathways using a small molecule inhibitor. Discussion of the immunologic/tumor vaccine approach against erbB2/EGFR are found in Reilly R T et al. (2000), Cancer Res. 60: 3569-3576; and Chen Y, Hu D, Eling D J, Robbins J, and Kipps T J. (1998), Cancer Res. 58: 1965-1971.

Members of the Bcl-2 family of proteins block apoptosis. Upregulation of bcl-2 has therefore been linked to chemoresistance. Studies have shown that the epidermal growth factor (EGF) stimulates anti-apoptotic members of the bcl-2 family (i.e., mcl-1). Therefore, strategies designed to downregulate the expression of bcl-2 in tumors have demonstrated clinical benefit and are now in Phase II/III trials, namely Genta's G3139 bcl-2 antisense oligonucleotide. Such proapoptotic strategies using the antisense oligonucleotide strategy for bcl-2 are discussed in Water J S et al. (2000), J. Clin. Oncol. 18: 1812-1823; and Kitada S et al. (1994), Antisense Res. Dev. 4: 71-79.

Cell cycle signalling inhibitors inhibit molecules involved in the control of the cell cycle. A family of protein kinases called cyclin dependent kinases (CDKs) and their interaction with a family of proteins termed cyclins controls progression through the eukaryotic cell cycle. The coordinate activation and inactivation of different cyclin/CDK complexes is necessary for normal progression through the cell cycle. Several inhibitors of cell cycle signalling are under development. For instance, examples of cyclin dependent kinases, including CDK2, CDK4, and CDK6 and inhibitors for the same are described in, for instance, Rosania et al, Exp. Opin. Ther. Patents (2000) 10(2):215-230.

Pharmaceutically active agents can cause injury to cardiovascular tissue for which the TPO receptor agonists of the invention are useful in treating the resultant cardiovascular disease.

Anti-neoplastic agents can cause injury to cardiovascular tissue for which the TPO receptor agonists of the invention are useful in treating the resultant cardiovascular disease.

Suitably, the anti-neoplasitc agent that can cause injury to cardiovascular tissue for which a TPO receptor agonist of the invention would be useful in treating the resultant cardiovascular disease is slected from: Doxorubicin, herceptin, Gleevac, Sprycel, Tasigna, Sutent, Nexavar, Avastin, Tykerb, Iressa, Tarceva, Erbitux and Panitumumab.

Suitably, the anti-neoplasitc therapy that can cause injury to cardiovascular tissue for which a TPO receptor agonist of the invention would be useful in treating the resultant cardiovascular disease is radiation therapy.

The present invention therefore provides a method of treating degenerative diseases/injuries, which comprises the administration of a therapeutically effective amount of a non-peptide TPO receptor agonist, suitably a compound of Formula (I), and/or a pharmaceutically acceptable salt, hydrate, solvate or ester thereof. The drug may be administered to a patient in need thereof by any conventional route of administration, including, but not limited to, intravenous, intramuscular, oral, subcutaneous, intradermal, and parenteral.

The non-peptide TPO receptor agonists of the present invention are incorporated into convenient dosage forms such as capsules, tablets, or injectable preparations. Solid or liquid pharmaceutical carriers are employed. Solid carriers include, starch, lactose, calcium sulfate dihydrate, terra alba, sucrose, talc, gelatin, agar, pectin, acacia, magnesium stearate, and stearic acid. Liquid carriers include syrup, peanut oil, olive oil, saline, and water. Similarly, the carrier or diluent may include any prolonged release material, such as glyceryl monostearate or glyceryl distearate, alone or with a wax. The amount of solid carrier varies widely but, preferably, will be from about 25 mg to about 1 g per dosage unit. When a liquid carrier is used, the preparation will be in the form of a syrup, elixir, emulsion, soft gelatin capsule, sterile injectable liquid such as an ampoule, or an aqueous or nonaqueous liquid suspension.

The pharmaceutical preparations are made following conventional techniques of a pharmaceutical chemist involving mixing, granulating, and compressing, when necessary, for tablet forms, or mixing, filling and dissolving the ingredients, as appropriate, to give the desired oral or parenteral products.

Doses of the pharmaceutically active compounds in a pharmaceutical dosage unit as described above will be an efficacious, nontoxic quantity preferably selected from the range of 0.001-100 mg/kg of active compound, preferably 0.002-50 mg/kg. When treating a human patient in need of a non-peptide TPO receptor agonist, the selected dose is administered preferably from 1-6 times daily, orally or parenterally. Preferred forms of parenteral administration include topically, rectally, transdermally, by injection and continuously by infusion. Oral dosage units for human administration preferably contain from 0.05 to 3500 mg, more preferably 0.1 to 3000 mg of active compound. Oral administration, which uses lower dosages is preferred. Parenteral administration, at high dosages, however, also can be used when safe and convenient for the patient.

Optimal dosages to be administered may be readily determined by those skilled in the art, and will vary with the particular non-peptide TPO receptor agonist in use, the strength of the preparation, the mode of administration, and the advancement of the disease condition. Additional factors depending on the particular patient being treated will result in a need to adjust dosages, including patient age, weight, diet, and time of administration.

The method of this invention of treating degenerative diseases/injuries in mammals, including humans, comprises administering to a subject in need thereof a therapeutically effective amount of a pharmaceutically active compound of the present invention.

The invention also provides for the use of a compound of Formula (I) in the manufacture of a medicament for use in the treatmet of degenerative diseases/injuries.

The invention also provides for the use of a compound of Formula (I) in the manufacture of a medicament for use in therapy.

The invention also provides for a pharmaceutical composition for use in the treatment of degenerative diseases/injuries which comprises a compound of Formula (I) and a pharmaceutically acceptable carrier.

The invention also provides for the use of a compound of Formula (II) in the manufacture of a medicament for use in the treatment of degenerative diseases/injuries.

The invention also provides for the use of a compound of Formula (II) in the manufacture of a medicament for use in therapy.

The invention also provides for a pharmaceutical composition for use in the treatment of degenerative diseases/injuries which comprises a compound of Formula (II) and a pharmaceutically acceptable carrier.

No unacceptable toxicological effects are expected when compounds of the invention are administered in accordance with the present invention.

In addition, the pharmaceutically active compounds of the present invention can be co-administered with further active ingredients, such as other compounds known to treat degenerative diseases/injuries or compounds known to have utility when used in combination with a non-peptide TPO receptor agonist.

Contemplated Equivalents—It will be appreciated by the person of ordinary skill in the art that the compounds of Formulas I and II may also exist in tautomeric forms. For example, in Formula I, the double bond that is drawn between the two nitrogen atoms exists between the lower nitrogen atom and the AR substituent. Tautomeric forms of the compounds of Formulas I and II are exemplified by the following Formula (IV):

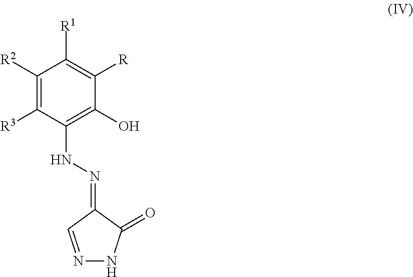

where the ‘R’ groups are as defined above. All such compounds are included in the scope of the invention and inherently included in the definition of the compounds of Formulas I and II.

Without further elaboration, it is believed that one skilled in the art can, using the preceding description, utilize the present invention to its fullest extent. The following Examples are, therefore, to be construed as merely illustrative and not a limitation of the scope of the present invention in any way.

### Experimental Details

### Example 1

### Capsule Composition

An oral dosage form for administering the present invention is produced by filing a standard two piece hard gelatin capsule with the ingredients in the proportions shown in Table I, below.

TABLE I

| INGREDIENTS | AMOUNTS |
|---|---|
| 4′-{N′-[1-(3,4-Dimethylphenyl)-3-methyl-5-oxo-1,5- | 25 mg |
| dihydropyrazol-4-ylidene]hydrazino}-3′-hydroxybiphenyl-4- | |
| carboxylic acid | |
| Lactose | 55 mg |
| Talc | 16 mg |
| Magnesium Stearate | 4 mg |

### Example 2

### Injectable Parenteral Composition

An injectable form for administering the present invention is produced by stirring 1.5% by weight of 4′-{N′-[1-(3,4-dimethylphenyl)-3-methyl-5-oxo-1,5-dihydropyrazol-4-ylidene]hydrazino}-3′-hydroxybiphenyl-3-carboxylic acid in 10% by volume propylene glycol in water.

### Example 3

### Tablet Composition

The sucrose, calcium sulfate dihydrate and a non-peptide TPO agonist, as shown in Table II below, are mixed and granulated in the proportions shown with a 10% gelatin solution. The wet granules are screened, dried, mixed with the starch, talc and stearic acid, then screened and compressed into a tablet.

TABLE II

| INGREDIENTS | AMOUNTS |
|---|---|
| 3′-{N′-[1-(3,4-dimethylphenyl)-3-methyl-5-oxo-1,5- | 20 mg |
| dihydropyrazol-4-ylidene]hydrazino}-2′-hydroxybiphenyl-3- | |
| carboxylic acid | |
| calcium sulfate dihydrate | 30 mg |
| sucrose | 4 mg |
| starch | 2 mg |
| talc | 1 mg |
| stearic acid | 0.5 mg |

While the preferred embodiments of the invention are illustrated by the above, it is to be understood that the invention is not limited to the precise instructions herein disclosed and that the right to all modifications coming within the scope of the following claims is reserved.

What is claimed is:

1. A method of treating cardiovascular disease in a human in need thereof which comprises the in vivo administration of a therapeutically effective amount of a compound selected from
3′-{N′-[1-(3,4-Dimethylphenyl)-3-methyl-5-oxo-1,5-dihydropyrazol-4-ylidene]hydrazino}-2′-hydroxybiphenyl-3-carboxylic acid,
or a pharmaceutically acceptable salt thereof, and
3-{N′-[1-(3,4-dimethylphenyl)-3-methyl-5-oxo-1,5-dihydropyrazol-4-ylidene]hydrazino}-2-hydroxy-3′-tetrazol-5-ylbiphenyl,
or a pharmaceutically acceptable salt thereof;
to such human.

2. A method according to claim 1 wherein the compound is 3′-{N′-[1-(3,4-Dimethylphenyl)-3-methyl-5-oxo-1,5-dihydropyrazol-4-ylidene]hydrazino}-2′-hydroxybiphenyl-3-carboxylic acid or a pharmaceutically acceptable salt thereof.

3. A method according to claim 1 wherein the compound is 3′-{N′-[1-(3,4-Dimethylphenyl)-3-methyl-5-oxo-1,5-dihydropyrazol-4-ylidene]hydrazino}-2′-hydroxybiphenyl-3-carboxylic acid bis-(monoethanolamine).

4. A method according to claim 3 wherein the cardiovascular disease is myocardial infarction.

5. A method according to claim 3 wherein the human is in need of treatment for repair of cardiovascular tissue.

6. A method according to claim 3 wherein the human is in need of tissue reparation for cardiovascular disorders.

7. A method according to claim 6 wherein the cardiovascular disorder occurred during cardiac bypass surgery.

8. A method according to claim 6 wherein the cardiovascular disorder occurred during heart surgery.

9. A method according to claim 8 wherein the heart surgery was heart transplant surgery.

10. A method according to claim 8 wherein the compound is administered prior to heart surgery.

11. A method according to claim 3 wherein the compound is administered orally.

12. A method according to claim 3 wherein the compound is administered parenterally.

13. A method according to claim 11 wherein the compound is administered in tablet form.

14. A method according to claim 3 wherein the cardiovascular disease is due to viral, fungal, microbial or parasitic infection.

15. A method according to claim 3 wherein the cardiovascular disease is due to surgical procedures.

16. A method according to claim 3 wherein the cardiovascular disease is due to treatment with antiviral or antibiotic agents.

17. A method according to claim 13 wherein the tablet contains an amount from 0.05 to 3500 mg of active compound.

18. A method according to claim 13 wherein the tablet contains an amount from 0.1 to 3000 mg of active compound.

19. A method according to claim 13 wherein the tablet contains 20 mg of active compound.

20. A method according to claim 3 wherein the cardiovascular disease is due to treatment with a pharmaceutically active agent.

21. A method according to claim 3 wherein the cardiovascular disease is due to treatment with an anti-neoplastic agent.

22. A method according to claim 21 wherein the cardiovascular disease is due to treatment with a chemotherapeutic agent.

23. A method according to claim 21 wherein the cardiovascular disease is due to treatment with a tyrosine kinase inhibiting compound.

24. A method according to claim 21 wherein the cardiovascular disease is due to treatment at least one compound selected from: Doxorubicin, herceptin, Gleevac, Sprycel, Tasigna, Sutent, Nexavar, Avastin, Tykerb, Iressa, Tarceva, Erbitux and Panitumumab.

25. A method according to claim 3 wherein the cardiovascular disease is due to treatment with radiation therapy.

26. A method according to claim 20 wherein the compound is administered in a tablet that contains an amount from 0.05 to 3500 mg of active compound.

27. A method according to claim 20 wherein the compound is administered in a tablet that contains an amount from 0.1 to 3000 mg of active compound.

28. A method according to claim 20 wherein the compound is administered in a tablet that contains 20 mg of active compound.

29. A method according to claim 21 wherein the compound is administered in a tablet that contains an amount from 0.05 to 3500 mg of active compound.

30. A method according to claim 21 wherein the compound is administered in a tablet that contains an amount from 0.1 to 3000 mg of active compound.

31. A method according to claim 21 wherein the compound is administered in a tablet that contains 20 mg of active compound.

32. A method according to claim 4 wherein the compound is administered in a tablet that contains an amount from 0.05 to 3500 mg of active compound.

33. A method according to claim 4 wherein the compound is administered in a tablet that contains an amount from 0.1 to 3000 mg of active compound.

34. A method according to claim 4 wherein the compound is administered in a tablet that contains 20 mg of active compound.

35. A method according to claim 3 wherein the compound is administered prior to heart surgery.

36. A method according to claim 3 wherein the compound is administered prior to treatment with a pharmaceutically active agent.

37. A method according to claim 3 wherein the compound is administered prior to treatment with an anti-neoplastic agent.

38. A method according to claim 3 wherein the compound is administered prior to treatment with a chemotherapeutic agent.

39. A method according to claim 3 wherein the compound is administered prior to treatment with a tyrosine kinase inhibiting compound.
